(19) United States
(12) Patent Application Publication
(10) Pub. No.: US 2025/0122167 A1
(43) Pub. Date: Apr. 17, 2025

(54) Pyrazine Compounds Useful in the Treatment of Parasitic Protozoal Infection

(71) Applicant: GLAXOSMITHKLINE INTELLECTUAL PROPERTY DEVELOPMENT LIMITED, Stevenage (GB)

(72) Inventor: Jorge Fernandez-Molina, Tres Cantos (ES)

(21) Appl. No.: 18/711,209

(22) PCT Filed: Nov. 21, 2022
(86) PCT No.: PCT/EP2022/082570

§ 371 Date: May 17, 2024

(30) Foreign Application Priority Data
Nov. 23, 2021 (EP) 21383056.5

(51) Int. Cl. C07D 401/04; A61K 31/497; A61K 45/06; A61P 33/06

(52) U.S. Cl. CPC C07D 401/04; A61K 31/497; A61K 45/06; A61P 33/06

(57) ABSTRACT

The present application relates to Compounds of Formula (I) and pharmaceutically acceptable salts or stereoisomers thereof, pharmaceutical compositions thereof, and their use in the treatment and prophylaxis of systemic infections, such as the treatment and prophylaxis of parasitic protozoal Infection, such as malaria, in particular infection by Plasmodium falciparum.

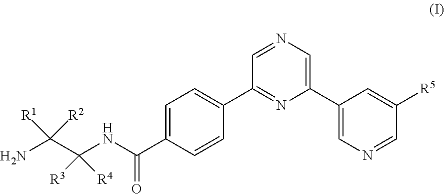

### FIELD OF THE INVENTION

The present application relates to compounds and pharmaceutically acceptable salts thereof, compositions thereof, and their use in the treatment and prophylaxis of systemic infections, such as the treatment and prophylaxis of parasitic protozoal infection, such as malaria, in particular infection by Plasmodium falciparum.

### BACKGROUND TO THE INVENTION

Parasitic infections are responsible for a wide variety of diseases of medical and veterinary importance, for example malaria in humans and coccidiosis in birds, fish, and mammals. Many of the diseases are life-threatening to the host and cause considerable economic loss in animal husbandry.

Malaria is a disease caused by protozoan parasites of the genus Plasmodium that infect and destroy red blood cells, leading to fever, severe anaemia, cerebral malaria, and if untreated, death. There are five species of Plasmodium parasite: falciparum, vivax, ovale, malariae, and knowlesi. Plasmodium falciparum is the most virulent. In 2019, there were an estimated 229 million people infected with malaria in 87 malaria endemic countries, and malarial disease was responsible for an estimated 409,000 deaths (World malaria report 2020:20 years of global progress and challenges. Geneva: World Health Organisation; 2020, Switzerland).

Effective oral preparations for the prophylaxis and treatment of malaria are known in the art. For example, known treatments/prophylaxis include artemisinin and artemisinin-based combination therapies (ACTs). ACTs are in fact the standard treatment for Plasmodium falciparum in uncomplicated cases (WHO Guidelines for malaria. Geneva: World Health Organization; 2021. Licence: CC BY-NC-SA 3.0 IGO). However, artermisinins are associated with delayed parasite clearance, which has been linked to less favourable clinical outcomes. A particular issue is emerging resistance to ACTs.

One approach to addressing this issue is to develop new therapeutic agents against with novel mechanisms of action. International patent application PCT/EP2016/074875 (published as WO 2017/067881) discloses pyrazine compounds and their use in the treatment of parasitic protozoal infections. However, there is therefore a need to develop new therapeutics that have improved physicochemical properties, whole cell potency, in vivo efficacy and in vitro predicted toxicities.

### SUMMARY OF THE INVENTION

In a first aspect, the present invention provides a compound according to Formula (I):

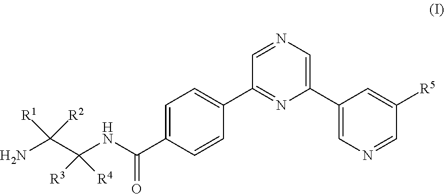

or a pharmaceutically acceptable salt or stereoisomer thereof, wherein:
R¹, R², R³, and R⁴ are each independently selected from the group consisting of H, halo, C₁-C₆ alkyl, and 3- to 7-membered cycloalkyl ring optionally containing a heteroatom selected from the group consisting of O, S, SO, SO₂, NH, and N—C₁-C₆ alkyl; or
R¹ and R² are together with the atom to which they are simultaneously attached form a 3-, 4-, 5-, 6-, or 7-membered cycloalkyl ring optionally containing a heteroatom selected from the group consisting of O, S, SO, SO₂, NH, and N—C₁-C₆ alkyl, wherein R³ and R⁴ are each independently selected from the group consisting of H, C₁-C₆ alkyl, and 3- to 7-membered cycloalkyl ring optionally containing a heteroatom selected from the group consisting of O, S, SO, SO₂, NH, and N—C₁-C₆ alkyl; or
R¹ and R³ are together with the atoms to which they are simultaneously attached form a 3-, 4-, 5-, 6-, or 7-membered cycloalkyl ring optionally containing a heteroatom selected from the group consisting of O, S, SO, SO₂, NH, and N—C₁-C₆ alkyl, wherein R² and R⁴ are each independently selected from the group consisting of H, C₁-C₆ alkyl, and 3- to 7-membered cycloalkyl ring optionally containing a heteroatom selected from the group consisting of O, S, SO, SO₂, NH, and N—C₁-C₆ alkyl; and wherein
R⁵ is selected from the group consisting of halo and C₁-C₆ alkyl.

In a second aspect, the present invention provides a compound selected from the group consisting of N-((1-aminocyclobutyl)methyl)-4-(6-(5-fluoropyridin-3-yl)pyrazin-2-yl)benzamide, N-(2-amino-2-methylpropyl)-4-(6-(5-fluoropyridin-3-yl)pyrazin-2-yl)benzamide, N-(2-aminocyclohexyl)-4-(6-(5-fluoropyridin-3-yl)pyrazin-2-yl)benzamide, and N-((1-aminocyclopropyl)methyl)-4-(6-(5-fluoropyridin-3-yl)pyrazin-2-yl)benzamide.

In a third aspect of the invention, there is provided a pharmaceutical composition comprising (a) a compound of Formula (I) or pharmaceutically acceptable salt or stereoisomer thereof; and (b) a pharmaceutically acceptable excipient.

In a fourth aspect of the invention, there is provided a compound of Formula (I) or a pharmaceutically acceptable salt or stereoisomer thereof for use in treating a parasitic protozoal infection.

In a fifth aspect of the invention, there is provided the use of the compound Formula (I) or the pharmaceutical composition including the Compound of Formula (I), in the manufacture of a medicament for treating a parasitic protozoal infection.

In a sixth aspect, the present invention provides a method of treating a parasitic protozoal infection in a human comprising administering to the human a therapeutically effective amount of the compound of Formula (I) or pharmaceutically acceptable salt or stereoisomer thereof, or the pharmaceutical composition.

In a seventh aspect, the present invention provides a combination comprising (a) the compound of Formula (I) or a pharmaceutically acceptable salt or stereoisomer thereof, and (b) at least one other anti-malarial agent.

In an eighth aspect, the present invention provides a method of treating a parasitic protozoal infection in a human comprising administering to the human a therapeutically effective amount of the combination.

### BRIEF DESCRIPTION OF THE FIGURES/DRAWINGS

The invention is further described by reference to the accompanying figures/drawings, which are non-limiting.

FIG. 1 shows an X-ray powder diffraction pattern of N-((1-aminocyclobutyl)methyl)-4-(6-(5-fluoropyridin-3-yl)pyrazin-2-yl)benzamide (Example 4).

FIG. 2 shows an X-ray powder diffraction pattern of N-((1S,2R)-2-aminocyclohexyl)-4-(6-(5-fluoropyridin-3-yl)pyrazin-2-yl)benzamide (Example 10).

### DETAILED DESCRIPTION OF THE EMBODIMENTS

### Statement of the Invention

As described above, in one aspect of the invention, there is provided a compound according to Formula (I):

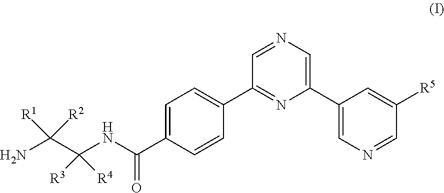

or a pharmaceutically acceptable salt or stereoisomer thereof, wherein:
R¹, R², R³, and R⁴ are each independently selected from the group consisting of H, halo, C₁-C₆ alkyl, and 3- to 7-membered cycloalkyl ring optionally containing a heteroatom selected from the group consisting of O, S, SO, SO₂, NH, and N—C₁-C₆ alkyl; or
R¹ and R² are together with the atom to which they are simultaneously attached form a 3-, 4-, 5-, 6-, or 7-membered cycloalkyl ring optionally containing a heteroatom selected from the group consisting of O, S, SO, SO₂, NH, and N—C₁-C₆ alkyl, wherein R³ and R⁴ are each independently selected from the group consisting of H, C₁-C₆ alkyl, and 3- to 7-membered cycloalkyl ring optionally containing a heteroatom selected from the group consisting of O, S, SO, SO₂, NH, and N—C₁-C₆ alkyl; or
R¹ and R³ together with the atoms to which they are simultaneously attached form a 3-, 4-, 5-, 6-, or 7-membered cycloalkyl ring optionally containing a heteroatom selected from the group consisting of O, S, SO, SO₂, NH, and N—C₁-C₆ alkyl, wherein R² and R⁴ are each independently selected from the group consisting of H, C₁-C₆ alkyl, and 3- to 7-membered cycloalkyl ring optionally containing a heteroatom selected from the group consisting of O, S, SO, SO₂, NH, and N—C₁-C₆ alkyl; and wherein
R⁵ is selected from the group consisting of halo and C₁-C₆ alkyl.

In one embodiment, R⁵ is halo. In one embodiment, R⁵ is fluoro (F).

In one embodiment, R⁵ is C₁-C₆ alkyl. In one embodiment, R⁵ is methyl.

In one embodiment, R³ is H. In one embodiment, R³ is H and R⁵ is halo, in particular fluoro.

In one embodiment, R³ is H and R⁵ is C₁-C₆ alkyl, in particular methyl.

In one embodiment, R³ and R⁴ are H. In one embodiment, R³ and R⁴ are H, and R⁵ is halo, in particular fluoro. In one embodiment, R³ and R⁴ are H, and R⁵ is C₁-C₆ alkyl, in particular methyl.

In one embodiment, R¹ and R² together with the atom to which they are simultaneously attached form a 3-, 4-, 5-, 6-, or 7-membered cycloalkyl ring. In one embodiment, R¹ and R² are together with the atom to which they are simultaneously attached form a 3-, 4-, 5-, 6-, or 7-membered cycloalkyl ring, in particular a 4-membered cycloalkyl ring, and R⁵ is halo, in particular fluoro. R¹ and R² are together with the atom to which they are simultaneously attached form a 3-, 4-, 5-, 6-, or 7-membered cycloalkyl ring, and R⁵ is C₁-C₆ alkyl, in particular methyl. In one embodiment, R¹ and R² are together with the atom to which they are simultaneously attached form a 3-, 4-, 5-, 6-, or 7-membered cycloalkyl ring, and R³ is hydrogen. In one embodiment, R¹ and R² are together with the atom to which they are simultaneously attached form a 3-, 4-, 5-, 6-, or 7-membered cycloalkyl ring, and R³ and R⁴ are hydrogen. In one embodiment, R¹ and R² are together with the atom to which they are simultaneously attached form a 3-, 4-, 5-, 6-, or 7-membered cycloalkyl ring, in particular a 4-membered cycloalkyl ring, R³ and R⁴ are hydrogen, and R⁵ is halo, in particular fluoro. In one embodiment, R¹ and R² are together with the atom to which they are simultaneously attached form a 3-, 4-, 5-, 6-, or 7-membered cycloalkyl ring, and R³ and R⁴ are hydrogen. In one embodiment, R¹ and R² are together with the atom to which they are simultaneously attached form a 3-, 4-, 5-, 6-, or 7-membered cycloalkyl ring, R³ and R⁴ are hydrogen, and R⁵ is C₁-C₆ alkyl, in particular methyl.

In one embodiment, the compound of Formula (I) is N-((1-aminocyclobutyl)methyl)-4-(6-(5-fluoropyridin-3-yl)pyrazin-2-yl)benzamide

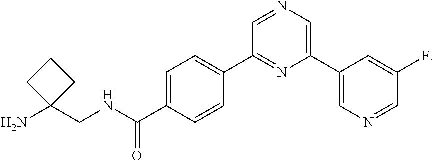 [SMILES: NC1(CNC(=O)c2ccc(-c3cncc(-c4cncc(F)c4)n3)cc2)CCC1]

In one embodiment, the compound of Formula (I) is in the form of a free base of N-((1-aminocyclobutyl)methyl)-4-(6-(5-fluoropyridin-3-yl)pyrazin-2-yl)benzamide.

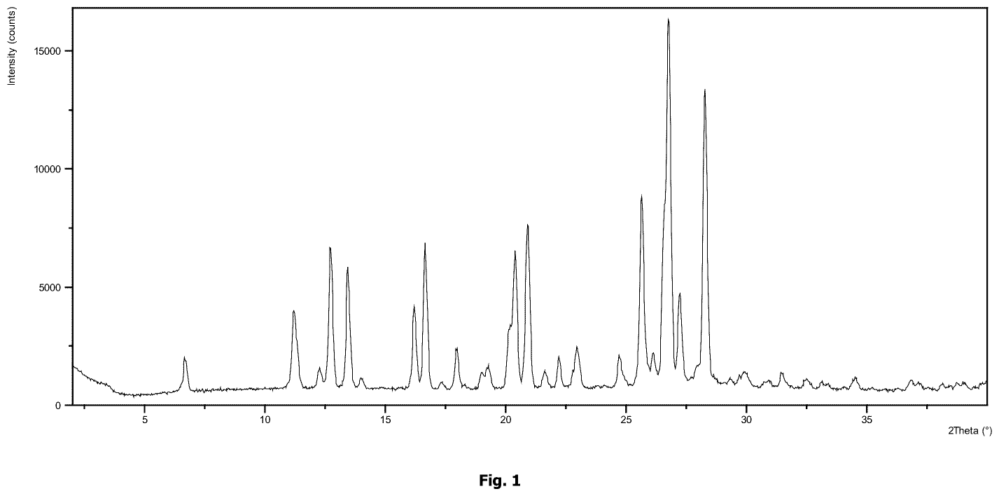

In one embodiment, the compound of Formula (I) is in the form of a free base of N-((1-aminocyclobutyl)methyl)-4-(6-(5-fluoropyridin-3-yl)pyrazin-2-yl)benzamide which has:
i) an X-ray powder diffraction pattern (XRPD) substantially as shown in FIG. 1; and/or
ii) an X-ray powder diffraction pattern (XRPD) with specific peaks at 20 values, +0.1° 20 experimental error, of 6.7, 11.2, 12.7, 13.4, 16.2, 16.6, 17.9, 20.9, 26.7, and 28.2 degrees.

In one embodiment, the compound of Formula (I) is N-((1-aminocyclobutyl)methyl)-4-(6-(5-fluoropyridin-3-yl)pyrazin-2-yl)benzamide is a pharmaceutically acceptable sulfuric acid salt.

In one embodiment, the compound of Formula (I) is in a pharmaceutically acceptable dihydrochloride.

In one embodiment, the compound of Formula (I) or pharmaceutically acceptable salt or stereoisomer thereof is N-((1-aminocyclopropyl)methyl)-4-(6-(5-fluoropyridin-3-yl)pyrazin-2-yl)benzamide

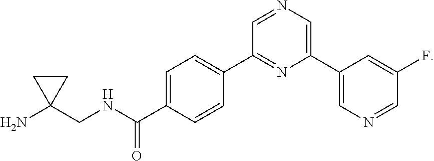 [SMILES: NC1(CNC(=O)c2ccc(-c3cncc(-c4cncc(F)c4)n3)cc2)CC1]

In one embodiment, R¹ and R² are each C₁-C₆ alkyl. In one embodiment, R¹ and R² are each C₁-C₆ alkyl, and R⁵ is halo, in particular fluoro. In one embodiment, R¹ and R² and R⁵ are each C₁-C₆ alkyl. In one embodiment, R¹ and R² are each C₁-C₆ alkyl, and R³ is H. In one embodiment, R¹ and R² are each C₁-C₆ alkyl, and R³ and R⁴ are each H. In one embodiment, R¹ and R² are each C₁-C₆ alkyl, R⁵ is halo, in particular fluoro, and R³ is H. In one embodiment, R¹ and R² are each C₁-C₆ alkyl, in particular each methyl, R⁵ is halo, in particular fluoro, and R³ and R⁴ are each H. In one embodiment, R¹ and R² and R⁵ are each C₁-C₆ alkyl, and R³ is H. In one embodiment, R¹ and R² and R⁵ are each C₁-C₆ alkyl, and R³ and R⁴ are each H.

In one embodiment, the compound of Formula (I) is N-(2-amino-2-methylpropyl)-4-(6-(5-fluoropyridin-3-yl)pyrazin-2-yl)benzamide

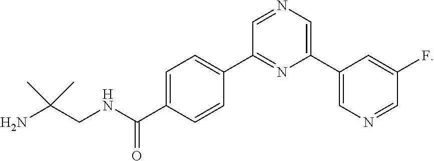 [SMILES: CC(C)(N)CNC(=O)c1ccc(-c2cncc(-c3cncc(F)c3)n2)cc1]

In one embodiment, R¹ and R³ together with the atoms to which they are simultaneously attached form a 3-, 4-, 5-, 6-, or 7-membered cycloalkyl ring. In one embodiment, R¹ and R³ together with the atoms to which they are simultaneously attached form a 3-, 4-, 5-, 6-, or 7-membered cycloalkyl ring, in particular a 6-membered cycloalkyl ring, and R⁵ is halo, in particular fluoro. In one embodiment, R¹ and R³ together with the atoms to which they are simultaneously attached form a 3-, 4-, 5-, 6-, or 7-membered cycloalkyl ring, and R⁵ is C₁-C₆ alkyl, in particular methyl.

In one embodiment, the compound of Formula (I) is N-(2-aminocyclohexyl)-4-(6-(5-fluoropyridin-3-yl)pyrazin-2-yl)benzamide

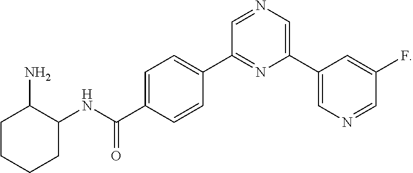 [SMILES: NC1CCCCC1NC(=O)c1ccc(-c2cncc(-c3cncc(F)c3)n2)cc1]

In one embodiment, the compound of Formula (I) is N-(2-aminocyclohexyl)-4-(6-(5-fluoropyridin-3-yl)pyrazin-2-yl)benzamide in the form of a free base.

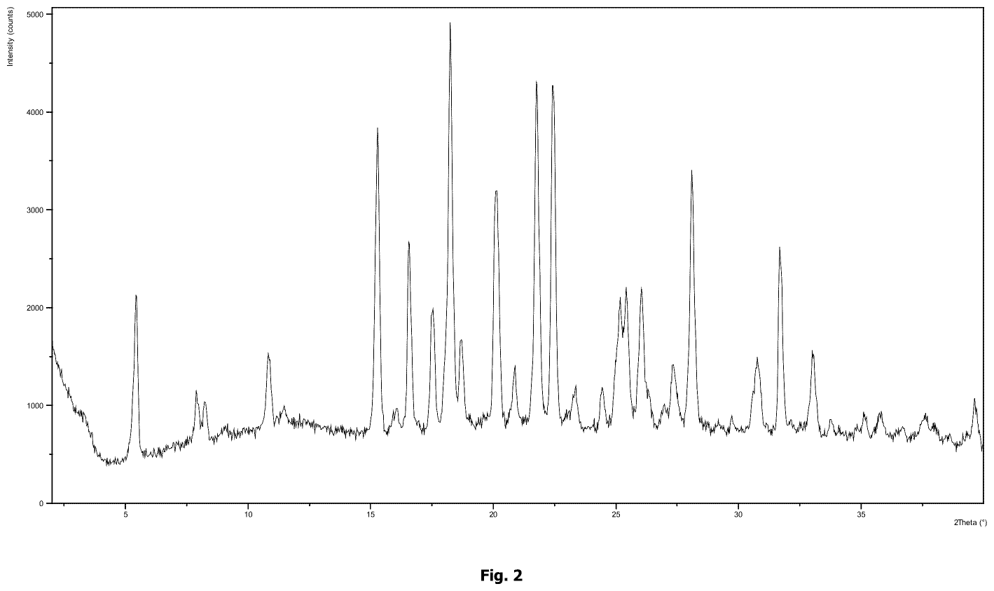

In one embodiment, the compound of Formula (I) is N-(2-aminocyclohexyl)-4-(6-(5-fluoropyridin-3-yl)pyrazin-2-yl)benzamide in the form of a free base which has:
i) an X-ray powder diffraction pattern (XRPD) substantially as shown in FIG. 2; and/or
ii) an X-ray powder diffraction pattern (XRPD) with specific peaks at 20 values, ±0.1° 2θ experimental error, of 5.4, 10.8, 15.3, 16.6, 18.2, 20.1, 21.8, 22.4, 28.1, and 31.7 degrees.

In each embodiment in which the compound of Formula (I) is N-(2-aminocyclohexyl)-4-(6-(5-fluoropyridin-3-yl)pyrazin-2-yl)benzamide, the compound may be N-((1S,2R)-2-aminocyclohexyl)-4-(6-(5-fluoropyridin-3-yl)pyrazin-2-yl)benzamide.

In one embodiment, the compound of Formula (I) or pharmaceutically acceptable salt or stereoisomer thereof is selected from the group consisting of N-((1-aminocyclobutyl)methyl)-4-(6-(5-fluoropyridin-3-yl)pyrazin-2-yl)benzamide, N-(2-amino-2-methylpropyl)-4-(6-(5-fluoropyridin-3-yl)pyrazin-2-yl)benzamide, N-(2-aminocyclohexyl)-4-(6-(5-fluoropyridin-3-yl)pyrazin-2-yl)benzamide, and N-((1-aminocyclopropyl)methyl)-4-(6-(5-fluoropyridin-3-yl)pyrazin-2-yl)benzamide.

In one embodiment, the invention provides a pharmaceutically acceptable salt of N-((1-aminocyclobutyl)methyl)-4-(6-(5-fluoropyridin-3-yl)pyrazin-2-yl)benzamide, N-(2-amino-2-methylpropyl)-4-(6-(5-fluoropyridin-3-yl)pyrazin-2-yl)benzamide, N-(2-aminocyclohexyl)-4-(6-(5-fluoropyridin-3-yl)pyrazin-2-yl)benzamide, or N-((1-aminocyclopropyl)methyl)-4-(6-(5-fluoropyridin-3-yl)pyrazin-2-yl)benzamide.

### Definitions

As used herein, the term “alkyl” represents a saturated, straight, or branched hydrocarbon group. The term “C₁-C₆ alkyl” refers to an alkyl group containing from 1 to 6 carbon atoms. Example alkyls include, but are not limited to, methyl, ethyl, n-propyl, isopropyl, n-butyl, isobutyl, s-butyl, t-butyl, n-pentyl, isopentyl, neopentyl, tert-pentyl, sec-pentyl, 3-pentyl, and sec-isopentyl.

The term “halogen” represents a chloro, iodo, bromo, or fluoro group.

The term “a compound of the invention” means any one of the compounds of the invention as defined above. Specifically, the term as used herein includes but is not limited to a compound of Formula (I) or a pharmaceutically acceptable salt or stereoisomer thereof and is a reference to any one of the Formulas described herein.

It will further be understood that the compounds of the invention, such as a compound of Formula (I) may exist in different tautomeric forms. Tautomers refer to isomeric forms of a compound that are in equilibrium with each other. The concentration of the isomeric forms will depend on the environment that the compound is in.

The term “pharmaceutically acceptable” refers to those compounds (including salts), materials, compositions, and dosage forms which are suitable for use contact with the tissues of humans or animals without excessive toxicity, irritation, or other side effect/complication.

In one aspect, the invention provides pharmaceutically acceptable salts of compounds of Formula (I). Pharmaceutically acceptable salts include but are not limited to those described in Berge, J. Pharm. Sci., 1977, 66, 1-19, or those listed in P H Stahl and C G Wermuth, Handbook of Pharmaceutical Salts: Properties, Selection and Use, Second Edition, John Wiley & Sons, March 2011.

Where the compound functionality allows, suitable pharmaceutically acceptable salts of a compound of Formula (I) can be formed, which include acid or base addition salts. Acid addition salts may be formed by reaction with the appropriate acid, optionally in a suitable solvent such as an organic solvent, to give the salt which can be isolated by crystallisation and filtration. Base addition salts may be formed by reaction with the appropriate base, optionally in a suitable solvent such as an organic solvent, to give the salt which can be isolated by crystallisation and filtration.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (W-benzylphenethylamine), benzathine (N,N′dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, f-butylamine, and zinc.

In one embodiment, the invention provides a pharmaceutically acceptable salt of the compound of Formula (I) selected from a ditrifluoroacetic acid salt of N-((1-aminocyclobutyl)methyl)-4-(6-(5-fluoropyridin-3-yl)pyrazin-2-yl)benzamide

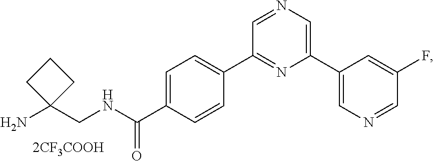 [SMILES: NC1(CNC(=O)c2ccc(-c3cncc(-c4cncc(F)c4)n3)cc2)CCC1]

a 2:1 hydrochloric acid salt of N-((1-aminocyclobutyl)methyl)-4-(6-(5-fluoropyridin-3-yl)pyrazin-2-yl)benzamide

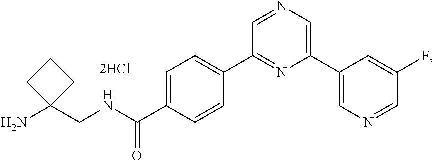 [SMILES: NC1(CNC(=O)c2ccc(-c3cncc(-c4cncc(F)c4)n3)cc2)CCC1]

and
a sulfuric acid salt of N-((1-aminocyclobutyl)methyl)-4-(6-(5-fluoropyridin-3-yl)pyrazin-2-yl)benzamide

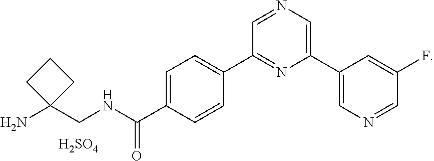

In one embodiment, the invention provides a pharmaceutically acceptable salt of the compound of Formula (I) which is a 2:1 hydrochloric acid salt of N-((1-aminocyclopropyl)methyl)-4-(6-(5-fluoropyridin-3-yl)pyrazin-2-yl)benzamide

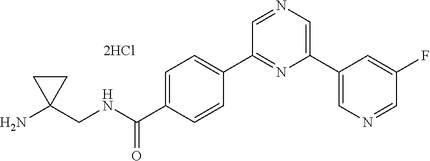 [SMILES: NC1(CNC(=O)c2ccc(-c3cncc(-c4cncc(F)c4)n3)cc2)CC1]

a 2:1 ditrifluoroacetic acid salt of N-((1-aminocyclopropyl)methyl)-4-(6-(5-fluoropyridin-3-yl)pyrazin-2-yl)benzamide

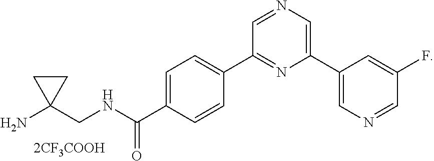 [SMILES: NC1(CNC(=O)c2ccc(-c3cncc(-c4cncc(F)c4)n3)cc2)CC1]

In one embodiment, the invention provides a pharmaceutically acceptable salt of the compound of Formula (I) which is a ditrifluoroacetic acid salt of N-(2-amino-2-methylpropyl)-4-(6-(5-fluoropyridin-3-yl)pyrazin-2-yl)benzamide

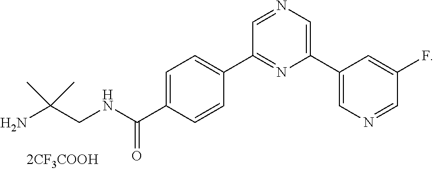 [SMILES: CC(C)(N)CNC(=O)c1ccc(-c2cncc(-c3cncc(F)c3)n2)cc1]

In one embodiment, the invention provides a pharmaceutically acceptable salt of the compound of Formula (I) which is selected from:
a 2:1 hydrochloride salt of N-(2-aminocyclohexyl)-4-(6-(5-fluoropyridin-3-yl)pyrazin-2-yl)benzamide

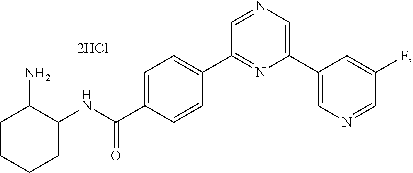 [SMILES: NC1CCCCC1NC(=O)c1ccc(-c2cncc(-c3cncc(F)c3)n2)cc1]

and a 2:1 trifluoroacetic acid salt of N-(2-aminocyclohexyl)-4-(6-(5-fluoropyridin-3-yl)pyrazin-2-yl)benzamide

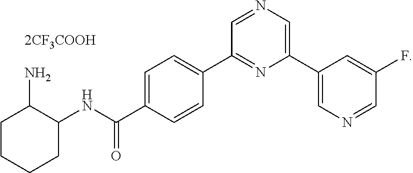 [SMILES: NC1CCCCC1NC(=O)c1ccc(-c2cncc(-c3cncc(F)c3)n2)cc1]

The invention includes within its scope all possible stoichiometric and non-stoichiometric forms of the salts of compounds of Formula (I). As used herein, the term “therapeutically effective amount” means any amount which, as compared to a corresponding human subject who has not received such amount, results in improved treatment, healing, prevention, or amelioration of a disease, disorder, or side effect, or a decrease in the rate of advancement of a disease or disorder.

An appropriate “therapeutically effective amount” will depend upon a number of factors including, for example, the age and weight of the human subject, the precise condition requiring treatment and its severity, the nature of the formulation, and the route of administration, and will ultimately be at the discretion of the attendant physician.

The compounds according to Formula (I) may contain one or more asymmetric centres (also referred to as a chiral centres) and may, therefore, exist as individual enantiomers, diastereomers, or other stereoisomeric forms, or as mixtures thereof. Chiral centres, such as chiral carbon atoms, may also be present in a substituent such as an alkyl group. Where the stereochemistry of a chiral centre present in Formula (I), or in any chemical structure illustrated herein, is not specified, the structure is intended to encompass any stereoisomer and all mixtures thereof. Thus, compounds according to Formula (I) containing one or more chiral centres may be used as racemic modifications including racemic mixtures and racemates, enantiomerically-enriched mixtures, or as enantiomerically-pure individual stereoisomers.

For solvates of the compounds of the invention, or salts thereof, that are in crystalline form, the skilled artisan will appreciate that pharmaceutically acceptable solvates may be formed wherein solvent molecules are incorporated into the crystalline lattice during crystallization. Solvates may involve non-aqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The invention also includes various deuterated forms of the compound of Formula (I) respectively or a pharmaceutically acceptable salt or stereoisomer thereof. Each available hydrogen atom attached to a carbon atom may be independently replaced with a deuterium atom. A person of ordinary skill in the art will know how to synthesize deuterated forms of the compound of Formula (I) or pharmaceutically acceptable salt or stereoisomer thereof of the present invention. For example, deuterated materials, such as alkyl groups may be prepared by conventional techniques.

The present invention also includes isotopically-labelled compounds which are identical to those recited in Formula (I) as defined herein, respectively, but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention or pharmaceutically acceptable salts or stereoisomers thereof that contain the aforementioned isotopes and/or other isotopes of other atoms are within the scope of the present invention. Isotopically labelled compounds of the present invention, for example those into which radioactive isotopes such as ³H or ¹⁴C have been incorporated, are useful in drug and/or substrate tissue distribution assays. Tritiated, i.e. ³H, and carbon-14, i.e. ¹⁴C, isotopes are particularly preferred for their ease of preparation and detectability. ¹¹C and ¹⁸F isotopes are particularly useful in PET (positron emission tomography).

Because the compounds of the present invention are intended for use in pharmaceutical compositions it will readily be understood that they are each provided in substantially pure form, for example at least 60% pure, in some aspects at least 75% pure, in some aspects at least 85% pure, and in other aspects at least 90% or 95% pure, especially at least 98% pure (% are on a weight for weight basis). Impure preparations of the compounds may be used for preparing the more pure forms used in the pharmaceutical compositions.

Compounds of Formula (I) or pharmaceutically acceptable salts or stereoisomers thereof may exist as solids or liquids, both of which are included in the invention. In the solid state, compounds of Formula (I) or pharmaceutically acceptable salts or stereoisomers thereof may exist as either amorphous material or in crystalline form, or as a mixture thereof. Compounds of Formula (I) or pharmaceutically acceptable salts or stereoisomers thereof may exist in solvated form and may be formed when solvent molecules are incorporated into the crystalline lattice during crystallisation. Solvates may involve non-aqueous solvents such as ethanol, isopropanol, dimethylsulfoxide (DMSO), acetic acid, ethanolamine, and ethyl acetate, or they may involve water as the solvent that is incorporated into the crystalline lattice. Solvates wherein water is the solvent that is incorporated are typically referred to as “hydrates”. Therefore, in embodiment, the present invention provides solvates of a compound of Formula (I), for example hydrates.

As used herein, the term “optionally” means that the subsequently described event(s) may or may not occur, and include both event(s) that occur and event(s) that do not occur. For example, when used in connection with the term “substituted”, i.e. “optionally substituted”, it means that the subsequently described substituents may be present or not present.

As used herein, the term “treatment” means: (1) the amelioration or prevention of the condition being treated or one or more of the biological manifestations of the condition being treat, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, an “effective amount” or a “safe and effective amount” means an amount of the compound sufficient to significantly induce a positive modification in the condition to be treated but low enough to avoid serious side effects (at a reasonable benefit/risk ratio) within the scope of sound medical judgement.

### Methods of Use

In one aspect, the invention relates to a compound of Formula (I), or a pharmaceutically acceptable salt or stereoisomer thereof, for use in therapy.

Compounds of Formula (I) or a pharmaceutically acceptable salt or stereoisomer thereof can be useful in the treatment of certain parasitic infections such as parasitic protozoal infections by the malarial parasite Plasmodium falciparum, species of Eimeria, Pneumocytis carinii, Trypanosoma cruzi, Trypanosoma brucei or Leishmania donovani.

In particular, the compound of Formula (I) or pharmaceutically acceptable salt or stereoisomer thereof can be useful for treatment of infection by Plasmodium falciparum. Accordingly, the invention is directed to methods of treating such infections. Alternatively, the compounds of Formula (I) or pharmaceutically acceptable salts or stereoisomer thereof can be useful for the treatment of infection by Plasmodium species other than Plasmodium falciparum causing human malaria. For example, the compounds of Formula (I) or pharmaceutically acceptable salts or stereoisomer thereof can be useful for the treatment of infection by Plasmodium Vivax, i.e., malaria caused by infection by Plasmodium vivax.

In one embodiment, the invention relates to a compound of Formula (I) or a pharmaceutically acceptable salt or stereoisomer thereof for use in the treatment of a parasitic protozoal infection. In a particular embodiment, said protozoal infection is malaria or infection by Plasmodium falciparum. In one embodiment, the invention relates to a compound of Formula (I) or a pharmaceutically acceptable salt or stereoisomer thereof for use in the treatment of malaria resulting from infection by Plasmodium falciparum.

In another aspect of the invention, there is provided a method for the treatment of a parasitic protozoal infection, comprising administering a pharmaceutically effective amount of a compound of Formula (I) or a pharmaceutically acceptable salt or stereoisomer thereof to a human in need thereof. In one embodiment, said parasitic protozoal infection is malaria or infection by Plasmodium falciparum.

In another aspect of the invention, there is provided the use of a compound of Formula (I) or a pharmaceutically acceptable salt or stereoisomer thereof in the manufacture of a medicament for the treatment of a parasitic protozoal infection. In one embodiment, said parasitic protozoal infection is malaria or infection by Plasmodium falciparum. Accordingly, a compound of Formula (I) or a pharmaceutically acceptable salt or stereoisomer thereof may be used in the treatment of malaria. Therefore, the invention also relates to a method for the treatment of malaria comprising administering a pharmaceutically effective amount of a compound of Formula (I) or a pharmaceutically acceptable salt or stereoisomer thereof to a human in need thereof. In addition, the invention relates to the use of a compound of Formula (I) or a pharmaceutically acceptable salt or stereoisomer thereof in the manufacture of a medicament for the treatment of malaria.

It will be appreciated by those skilled in the art that references herein to treatment refer to the treatment of established conditions, such as malaria. However, compounds of the invention may also be useful in the prevention of such diseases, such as in the prevention of malaria. Thus, in one embodiment, there is provided the treatment or prevention of a disease such as malaria. In another embodiment, there is provided the treatment of a disease such as malaria. In a further embodiment, there is provided the prevention of a disease such as malaria.

In one embodiment, the malaria is multi-drug resistant malaria. Therefore, in one embodiment, a compound of Formula (I) or a pharmaceutically acceptable salt or stereoisomer thereof may be useful in the treatment of sensitive and/or multi-drug resistant malaria.

In one embodiment, the compound of Formula (I) or pharmaceutically acceptable salt or stereoisomer thereof is for use in the treatment of a parasitic protozoal infection that is resistant to artemisinin combination therapy. In one embodiment, the compound of Formula (I) or pharmaceutically acceptable salt or stereoisomer thereof is for use in the treatment of malaria that is resistant to artemisinin combination therapy.

### Pharmaceutical Compositions

The compounds of Formula (I) and pharmaceutically acceptable salts and stereoisomer thereof will normally, but not necessarily, be formulated into pharmaceutical compositions prior to administration to a patient. Accordingly, in another aspect there is provided a pharmaceutical formulation comprising (a) a compound of Formula (I), or a pharmaceutically acceptable salt or stereoisomer thereof; and (b) a pharmaceutically acceptable excipient or carrier.

Suitable pharmaceutically acceptable excipients include the following types of excipients: binders, disintegrants, lubricants, glidants, granulating agents, coating agents, wetting agents, solvents, co-solvents, suspending agents, emulsifiers, sweeteners, flavouring agents, flavour masking agents, colouring agents, anticaking agents, humectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation. The carrier excipient must be “acceptable” in the sense of being compatible with the other ingredients of the formulation and not deleterious to the recipient thereof. Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company). For example, dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; and (5) inhalation such as aerosols and solutions.

In one aspect, the invention is directed to a solid or liquid oral dosage form such as a liquid, tablet, lozenge or a capsule, comprising a safe and effective amount of a compound of the invention and a carrier. The carrier may be in the form of a diluent or filler. Suitable diluents and fillers in general include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. A liquid dosage form will generally consist of a suspension or solution of the compound or pharmaceutically acceptable salt or stereoisomer in a liquid carrier for example, ethanol, olive oil, glycerine, glucose (syrup) or water (e.g. with an added flavouring, suspending, or colouring agent). Where the composition is in the form of a tablet or lozenge, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers or a semi solid e.g. mono di-glycerides of capric acid, Gelucire and Labrasol, or a hard-capsule shell e.g. gelatin. Where the composition is in the form of a soft-shell capsule e.g. gelatin, any pharmaceutical carrier routinely used for preparing dispersions or suspensions may be considered, for example aqueous gums or oils, and may be incorporated in a soft capsule shell.

Pharmaceutical compositions may be administered by any appropriate route, for example by the oral (including buccal or sublingual), inhaled, intranasal, topical (including buccal, sublingual or transdermal), parenteral (including subcutaneous, intramuscular, intravenous or intradermal) route. In particular, pharmaceutical compositions are administered via an oral route of administration. Pharmaceutical compositions may be presented in unit dose forms containing a predetermined amount of active ingredient per unit dose. In one aspect, the unit dosage compositions are those containing a daily dose or sub-dose, or an appropriate fraction thereof, of an active ingredient. Such unit doses may therefore be administered more than once a day. In one aspect, the unit dosage compositions are those containing a daily dose or sub-dose (for administration more than once a day), as herein above recited, or an appropriate fraction thereof, of an active ingredient.

An oral solid dosage form may further comprise an excipient in the form of a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise an excipient in the form of a disintegrant. Suitable disintegrants include crospovidone, sodium starch glycolate, croscarmellose, alginic acid, and sodium carboxymethyl cellulose. The oral solid dosage form may further comprise an excipient in the form of a lubricant. Suitable lubricants include stearic acid, magnesium stearate, calcium stearate, and talc.

In one embodiment, the compound of Formula (I) or pharmaceutically acceptable salt or stereoisomer thereof is prepared for administration by injection, either intramuscularly or subcutaneously. In one aspect, the present invention relates to an injectable composition comprising the compound of Formula (I) or pharmaceutically acceptable salt or stereoisomer thereof. Standard formulation and manufacturing techniques can be used to produce a suitable stable, sterile vehicle for injection containing the compound of Formula (I) or pharmaceutically acceptable salt or stereoisomer thereof of the present invention. In some aspects, the injectable pharmaceutical composition is a long-acting injectable composition and provides a controlled release of the compound of Formula (I) or pharmaceutically acceptable salt or stereoisomer thereof.

In one embodiment, the compound of Formula (I) or pharmaceutically acceptable salt or stereoisomer thereof is prepared for administration by injection, with the composition comprising the compound and a pharmaceutically acceptable excipient or carrier, such as Tween 20, PEG400 and/or mannitol. In one embodiment, the compound of Formula (I) or pharmaceutically acceptable salt or stereoisomer thereof is formulated with Tween 20, PEG400 and mannitol, and suitable as a long-acting injectable composition.

### Combinations

When a compound of Formula (I) or pharmaceutically acceptable salt or stereoisomer thereof is used in the treatment of malaria, or Plasmodium falciparum, it may be employed alone or in combination with at least one other therapeutic agent, such as at least one other anti-parasitic agents, for example an anti-malarial agent.

The present invention thus provides a combination of (a) a compound of Formula (I) or a pharmaceutically acceptable salt or stereoisomer thereof; and (b) at least one other anti-malarial agent. In an embodiment, the combination comprises one or two or three additional anti-malarial agents. For the avoidance of doubt, the at least one other anti-malarial agent is not a compound of Formula (I).

The at least one other anti-malarial agent is an agent in development, approved of recommended for the treatment of malaria.

The at least one other anti-malarial agent may be selected from chloroquine, mefloquine, primaquine, pyrimethamine, quinine, artemisinin, halofantrine, doxycycline, amodiaquine, atovaquone, tafenoquine, dapsone, proguanil, sulfadoxine, cycloguanil, fansidar, piperaquine, lumefantrine, artesunate, dihydroartemisinin, arthemeter, fosmidomycin and azithromycin.

In one embodiment, the at least one other anti-malarial agent is an artemisinin agent.

The at least one other anti-malarial agent may be tafenoquine.

In an embodiment, the additional anti-malarial agents are atovaquone and proguanil. The at least one other anti-malarial agent may also be selected from ferroquine, KAF156, cipargamin, DSM265, artemisone, artemisinin, artefenomel, MMV048, SJ733, P218, MMV253, PA92, DDD498, AN13762, DSM421, UCT947, ACT 451840, 6-chloro-7-methoxy-2-methyl-3-{4-[4-(trifluoromethoxy)phenoxy]phenyl}quinolin-4(1H)-one, 6-chloro-7-methoxy-2-methyl-3-(4-(4-(trifluoromethoxy)phenoxy)phenyl)quinolin-4(1H)-one, a pharmaceutically salt thereof, and a combination thereof. In one embodiment, the additional anti-malarial agent is 6-chloro-7-methoxy-2-methyl-3-{4-[4-(trifluoromethoxy)phenoxy]phenyl}quinolin-4(1H)-one, 6-chloro-7-methoxy-2-methyl-3-(4-(4-(trifluoromethoxy)phenoxy)phenyl)quinolin-4(1H)-one, a pharmaceutically salt thereof, or a combination thereof.

The at least one other anti-malarial agent may also be selected from OZ609, OZ277 and SAR97276.

In the treatment of Plasmodium falciparum infections, the at least one or two or three additional anti-malarial agents are selected as follows, wherein at least one of the anti-malarial agents is an artemisinin-based agent:
artesunate+sulfadoxine-pyrimethamine (SP)
artemether+lumefantrine
artesunate+amodiaquine
artesunate+mefloquine
dihydroartemisinin+piperaquine

The above combination treatments are known as artemisinin-based combination therapies (ACTs). The choice of ACT is usually based on the results of therapeutic efficacy studies against local strains of Plasmodium falciparum malaria.

In the treatment of Plasmodium vivax infections, an ACT may be used, as described above. Alternatively, the at least one other anti-malarial agent may be chloroquine, particularly in areas without chloroquine resistant Plasmodium vivax. In areas where resistant Plasmodium vivax has been identified, infections may be treated with an ACT, as described above.

The combinations may conveniently be presented for use in the form of a pharmaceutical composition or formulation. Therefore, also contemplated herein is a pharmaceutical composition comprising (a) a compound of Formula (I) or a pharmaceutically acceptable salt or stereoisomer thereof, as herein described, together with (b) at least one other anti-malaria agent and (c) one or more pharmaceutically acceptable excipients as herein described.

A compound of Formula (I) or pharmaceutically acceptable salt or stereoisomer thereof and at least one other therapeutic agent may be administered together or separately and, when administered separately, this may occur separately or sequentially in any order (by the same or by different routes of administration).

### Dosages

The amount of a compound of the invention or pharmaceutically acceptable salt or stereoisomer thereof and the further therapeutically active agent(s) and the relative timings of administration will be selected in order to achieve the desired combined therapeutic effect, and can be subject to the judgement of a health-care practitioner.

Typical amounts administered will be about 0.1 mg-1000 mg, in some aspects about 0.1 mg to about 500 mg. In one embodiment, the compound of Formula (1) or a pharmaceutically acceptable salt or stereoisomer thereof is administered in an amount of 200 mg.

The compounds of the invention may be administered orally in a range of doses, for example 0.1 to 5000 mg, or 1 to 1500 mg, 2 to 800 mg, or 5 to 500 mg, e.g. 2 to 200 mg or 10 to 1000 mg, particular examples of doses including 10, 20, 50, 80 mg, and 350 mg.

In one embodiment, the therapeutically effective single dose of the compound of Formula (1) is between about 10 to about 150 mg QD for three sequential days. In one embodiment, the compound of Formula (1) or a pharmaceutically acceptable salt or stereoisomer thereof is administered in an amount of between about 50 mg to about 85 mg QD for three sequential days. In one embodiment compound of Formula (1) or a pharmaceutically acceptable salt or stereoisomer thereof is administered in an amount of between about 10 mg to about 50 mg QD for three sequential days.

In one embodiment, a compound or pharmaceutically acceptable salt or stereoisomer thereof selected from the group consisting of N-((1-aminocyclobutyl)methyl)-4-(6-(5-fluoropyridin-3-yl)pyrazin-2-yl)benzamide, N-(2-amino-2-methylpropyl)-4-(6-(5-fluoropyridin-3-yl)pyrazin-2-yl)benzamide, N-(2-aminocyclohexyl)-4-(6-(5-fluoropyridin-3-yl)pyrazin-2-yl)benzamide, and N-((1-aminocyclopropyl)methyl)-4-(6-(5-fluoropyridin-3-yl)pyrazin-2-yl)benzamide is administered in an amount of about 0.1 mg-1000 mg. In one embodiment, a compound or pharmaceutically acceptable salt or stereoisomer thereof selected from the group consisting of N-((1-aminocyclobutyl)methyl)-4-(6-(5-fluoropyridin-3-yl)pyrazin-2-yl)benzamide, N-(2-amino-2-methylpropyl)-4-(6-(5-fluoropyridin-3-yl)pyrazin-2-yl)benzamide, N-(2-aminocyclohexyl)-4-(6-(5-fluoropyridin-3-yl)pyrazin-2-yl)benzamide, and N-((1-aminocyclopropyl)methyl)-4-(6-(5-fluoropyridin-3-yl)pyrazin-2-yl)benzamide is administered in an amount of about 200 mg.

Ultimately, however, the quantity of compound administered, and the type of composition used will be commensurate with the nature of the disease or physiological condition being treated and will be at the discretion of the physician.

Determination of effective dosages in this context is typically based on animal model studies followed up by human clinical trials and is guided by administration protocols that significantly reduce the occurrence or severity of targeted disease symptoms or conditions in the human. Suitable models in this regard include, for example, murine, rat, avian, porcine, feline, non-human primate, and other accepted animal model subjects known in the art. Alternatively, effective dosages can be determined using in vitro models (for example, whole cell assays that monitor the effect of various drugs on parasite growth rate). Using such models, only ordinary calculations and adjustments are required to determine an appropriate concentration and dose to administer a therapeutically effective amount of the compound (for example, amounts that are effective to elicit a desired immune response or alleviate one or more symptoms of a targeted disease).

### General Synthetic Routes

The compounds of the invention may be made by a variety of methods, which include those methods conventionally known in the field of chemistry. Any previously defined variable will continue to have the previously defined meaning unless otherwise indicated. Illustrative general synthetic methods are set out in the following schemes, and can be readily adapted to prepare the compounds of the invention. Specific compounds prepared according to the experimental procedure are disclosed in the Examples Section.

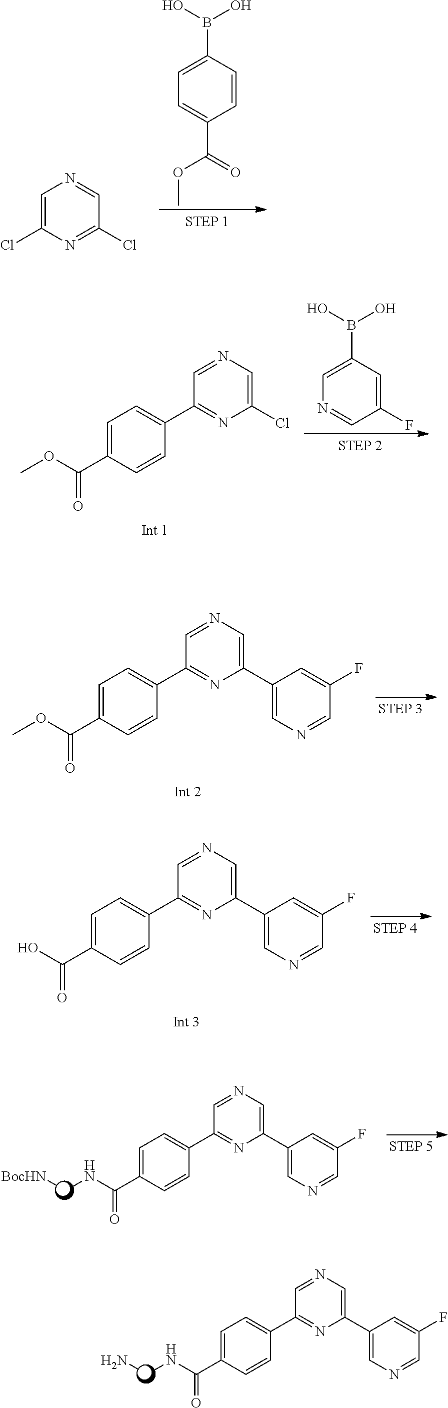

Steps 1 and 2 are Suzuki coupling reactions which can be carried out using the appropriate boronic acid or ester in the presence of a suitable catalyst such as PdCl₂(dppf) and a base such as sodium carbonate, in an adequate solvent such as isopropanol/water, and at a suitable temperature such as 120° C.
Step 3 is a de-esterification reaction that can be carried out using the appropriate conditions, such as LiOH·H₂O in an adequate mixture of solvents such as THF/H₂O.
Step 4 is a coupling reaction using the appropriate acid intermediate and the corresponding amine as a coupling partner, in the presence of coupling agent such as HATU and in the presence of a base such as DIPEA, in an adequate solvent as a DMF.
Step 5: is a deprotection, which can be carried out by a suitable acid such as 4M HCl in 1,4-dioxane.

### EXAMPLES

The invention will now be illustrated by way of the following non-limiting examples. While particular embodiments of the invention are described below a skilled artisan will appreciate that various changes and modifications can be made. References to preparations carried out in a similar manner to, or by the general method of, other preparations, may encompass variations in routine parameters such as time, temperature, work-up conditions, and minor changes in reagent amounts, etc.

In certain of the following intermediates and examples, starting materials are identified by reference to other intermediate or example numbers. This does not signify that the actual material from any particular intermediate or example was necessarily used in a subsequent step exemplified herein, but is used as a short-hand means of denoting the relevant compound.

Where materials were commercially available, this is indicated in parentheses after the compound name in capitals. Commercial reagents and solvents were used as received. All solvents used in the reaction were high purity grade or anhydrous grade. Proton nuclear magnetic resonance (¹H NMR) spectra were recorded, and chemical shifts are reported in parts per million (δ) downfield from the internal standard tetramethylsilane (TMS). Abbreviations for NMR data are as follows: s=single, d=doublet, t=triplet, q=quartet, m=multiplet, dd=doublet of doublets, dt=doublet of triplets, app=apparent, br=broad. Mass spectra were obtained using electrospray (ES) ionisation techniques. All temperatures are reported in degrees centigrade.

Where diastereomers are represented and the absolute stereochemistry is known, the stereocentre, i.e. the chiral carbon atom, is labelled with R or S.

### Abbreviations

AcOEt Ethyl acetate
ACN Acetonitrile
CDCl₃ deuterated chloroform
DCM Dichloromethane
DIPEA N,N-Diisopropylethylamine
DMF Dimethylformamide
DMSO dimethyl sulfoxide
EtOH Ethanol
h hour(s)
HATU Hexafluorophosphate Azabenzotriazole Tetramethyl Uronium
HCl hydrochloric acid
HPLC High Performance Liquid Chromatography
i-PrOH Isopropylalcohol
K₂CO₃ potassium carbonate
min minutes
MeOH methanol
NaOH Sodium hydroxide
Na₂CO₃ Sodium carbonate
NH₄Cl Ammonium chloride
RBF Round bottom flask
Rt Room temperature
SiO₂ Silicon dioxide
TBME or tBuOMe tert-butyl methyl ether
TFA trifluoroacetic acid

### Intermediate 1: Methyl 4-(6-chloropyrazin-2-yl)benzoate

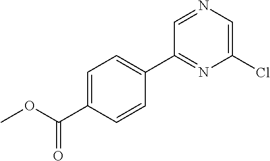 [SMILES: COC(=O)c1ccc(-c2cncc(Cl)n2)cc1]

Into a 100 mL RBF (4-(methoxycarbonyl)phenyl) boronic acid (Apollo Scientific, 1.0 g, 5.56 mmol), 2,6-dichloropyrazine (Combi-Blocks, 1.656 g, 11.11 mmol) and K₂CO₃ (Chempure, 2.304 g, 16.67 mmol) was added at room temperature. Then 1,2-Dimethoxyethane (30.0 mL) and water (10.0 mL) were added at same temperature. Then obtained reaction mixture was degassed with nitrogen for 15 minutes, followed by addition of PdCl₂(dppf)-CH₂Cl₂ adduct (Chempure, 0.159 g, 0.194 mmol) at room temperature. Then obtained reaction mixture was heated to 65° C. and stirred at same temperature for 16 hours. The reaction mixture was diluted with water (10 mL) and extracted with ethyl acetate (2×25 mL). The combined organic phase was dried over Na₂SO₄ (5 g), filtered and the filtrate was concentrated under reduced pressure to get a black color solid as crude.

The crude product was dissolved in 15 mL of DCM, adsorbed on 10 g of silica gel (230-400 mesh) and purified by Biotage isolera column chromatography (silica gel 230-400 mesh, 40 g snap, flow rate 30 mL/minute). The desired compound was eluted with 0-15% of ethyl acetate in pet. ether, collected fractions were pooled and concentrated under reduced pressure to get the titled compound (900 mg, 3.59 mmol, 64.5% yield) as a white solid.

¹H NMR (δ, ppm, CDCl₃): 9.38 (s, 1H), 8.84 (s, 1H), 8.29 (d, 2H), 8.12 (d, 2H), 3.91 (s, 3H). [ES MS] m/z: 249 (MH⁺).

### Intermediate 2: methyl 4-(6-(5-fluoropyridin-3-yl)pyrazin-2-yl)benzoate

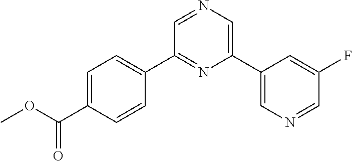 [SMILES: COC(=O)c1ccc(-c2cncc(-c3cncc(F)c3)n2)cc1]

Into a 10000 mL RBF methyl 4-(6-chloropyrazin)benzoate (250 g, 1005 mmol), (5-fluoropyridin-3-yl) boronic acid (COMBI-BLOCKS, 156 g, 1106 mmol) and K₂CO₃ (Chempure, 417 g, 3016 mmol) was added at room temperature. Then 1,4-dioxane (5000 mL) and water (1500.0 mL) were added at same temperature. The obtained reaction mixture was degassed with nitrogen for 15 minutes, followed by addition of PdCl₂(dppf)-CH₂Cl₂ adduct (Chempure, 28.7 g, 35.2 mmol) at room temperature. Then obtained reaction mixture was heated to 55° C. and stirred at same temperature for 3 hours. The reaction mixture was allowed to room temperature and kept for 4 hours at same temperature, solid was crashed out. The solid was filtered and washed with water (2000 mL), acetone (2500 mL) and followed by pet. ether (1500 mL), dried under reduced pressure to get methyl 4-(6-(5-fluoropyridin-3-yl)pyrazin-2-yl)benzoate (280.0 g, 897 mmol, 89% yield) as white solid.

¹H NMR (δ, ppm, DMSO-D6): 9.44 (s, 1H), 9.42 (s, 1H), 9.37 (s, 1H), 8.77 (d, 1H), 8.65-8.55 (m, 1H), 8.48 (d, 2H), 8.15 (d, 2H), 3.92 (s, 3H). [ES MS] m/z: 310 (MH⁺).

### Intermediate 3:4-(6-(5-fluoropyridin-3-yl)pyrazin-2-yl)benzoic acid

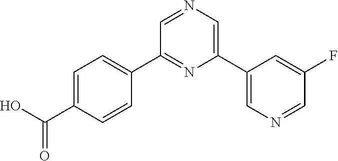 [SMILES: O=C(O)c1ccc(-c2cncc(-c3cncc(F)c3)n2)cc1]

To a stirred solution of methyl 4-(6-(5-fluoropyridin-3-yl)pyrazin-2-yl)benzoate (200.0 g, 647 mmol) in Tetrahydrofuran (2500 mL) at room temperature lithium hydroxide monohydrate was added (Chempure, 81 g, 1940 mmol) in Water (1000 mL). The obtained reaction mixture was stirred for 12 h at room temperature. The reaction mixture was concentrated under reduced pressure to get crude product as white solid. The crude product was diluted with water (500 mL) and pH was adjusted to 3-4 by using 1.5N HCl (550 mL) upon which white solid crashed out. The obtained reaction mixture was stirred for 15 min and solid obtained was filtered and washed with water (2500 mL), followed by acetone (1500 mL) and dried under reduced pressure to get the titled compound (187 g, 632 mmol, 98% yield) as a white solid.

¹H NMR (δ, ppm, DMSO-D6): 13.21 (bs, 1H), 9.43 (s, 1H), 9.42 (s, 1H), 9.38 (t, 1H), 8.77 (d, 1H), 8.62-8.58 (m, 1H), 8.45 (d, 2H), 8.13 (d, 2H). [ES MS] m/z: 296 (MH⁺).

### Intermediate 4: tert-butyl(1-((4-(6-(5-fluoropyridin-3-yl)pyrazin-2-yl)benzamido)methyl)cyclobutyl)carbamate

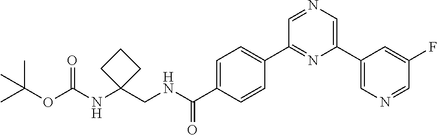 [SMILES: CC(C)(C)OC(=O)NC1(CNC(=O)c2ccc(-c3cncc(-c4cncc(F)c4)n3)cc2)CCC1]

To a suspension of 4-(6-(5-fluoropyridin-3-yl)pyrazin-2-yl)benzoic acid (20 g, 67.7 mmol) and DIPEA (ALDRICH, 59.2 ml, 339 mmol) in N,N-Dimethylformamide (DMF) (300 ml), HATU (FLUOROCHEM, 33.5 g, 88 mmol) was added (after 10 min stirring not solution). Neat powdered tert-butyl (1-(aminomethyl)cyclobutyl)carbamate (ENAMINE, 16.28 g, 81 mmol) was added and the resulting mixture was stirred at room temperature.

The reaction was diluted with AcOEt (100 ml) and 1N NH₄Cl was added until complete precipitation of white solid (500 ml). The mixture was filtered and rinsed with water (2×100 ml). The solid dried under vacuum to obtain the title compound (31 g, 64.9 mmol, 96%) as a white solid.

¹H NMR (δ, ppm, CDCl₃): 9.42 (d, 1H), 9.38 (t, 1H), 8.77 (d, 1H), 8.63-8.57 (m, 1H), 8.43 (d, 2H), 8.05 (d, 2H), 7.01 (br s, 1H), 3.58 (d, 2H), 2.35-2.20 (m, 2H), 2.13-2.01 (m, 2H), 1.87-1.85 (m, 2H), 1.40 (s, 9H). [ES MS] m/z: 478 (MH⁺).

### Example 1: N-((1-aminocyclobutyl)methyl)-4-(6-(5-fluoropyridin-3-yl)pyrazin-2-yl)benzamide, ditrifluoroacetic acid salt

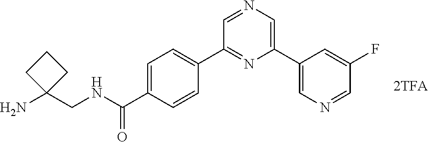 [SMILES: NC1(CNC(=O)c2ccc(-c3cncc(-c4cncc(F)c4)n3)cc2)CCC1]

To a solution of tert-butyl (1-((4-(6-(5-fluoropyridin-3-yl)pyrazin-2-yl)benzamido)methyl)cyclobutyl)carbamate (50 mg, 0.105 mmol) in dry dichloromethane (2 mL) under N₂ atmosphere, trifluoroacetic acid (1.000 mL) was added and the resulting mixture was stirred at room temperature. 30 min later, the reaction was diluted with DCM and concentrated under reduced pressure. The resulting crude was purified by preparative HPLC (sample loaded in 1.5 mL of MeOH, XBridge 19×150 mm, flow 17 ml/min, using as a mobile phase ACN and H₂O with 0.1% TFA), to give, after collecting and lyophilizing the appropriate fraction, 35 mg of aminocyclobutyl)methyl)-4-(6-(5-fluoropyridin-3-yl)pyrazin-2-yl)benzamide, ditrifluoroacetic acid salt (35 mg, 0.058 mmol, 55.2% yield) as a white solid.

¹H NMR (δ, ppm, DMSO-d₆): 9.43 (d, 1H), 9.39-9.36 (m, 1H), 8.95-8.88 (m, 1H), 8.78 (d, 1H), 8.65-8.56 (m, 1H), 8.46 (d, 2H), 8.10 (d, 2H), 8.07-7.98 (m, 2H), 3.67 (d, 2H), 2.28-2.10 (m, 4H), 1.99-1.76 (m, 2H). [ES MS] m/z: 378 (MH⁺).

### Example 2: N-((1-aminocyclobutyl)methyl)-4-(6-(5-fluoropyridin-3-yl)pyrazin-2-yl)benzamide, dihydrochloride

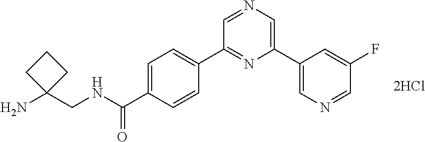 [SMILES: NC1(CNC(=O)c2ccc(-c3cncc(-c4cncc(F)c4)n3)cc2)CCC1]

To a suspension of tert-butyl (1-((4-(6-(5-fluoropyridin-3-yl)pyrazin-2-yl)benzamido)methyl)cyclobutyl)carbamate (100 mg, 0.209 mmol) in anhydrous 1-Propanol (2 mL) under N₂ atmosphere, 6N hydrochloric acid in iPrOH (FLUOROCHEM, 2 mL, 12.00 mmol) was added and the resulting mixture was stirred at room temperature. 2 h later, TBME was added and the resulting suspension was concentrated to give a cream-colored solid. This solid was treated with ACN and filtered and washed three times. The solid was dried under air stream to give 80 mg of titled compound (80 mg, 0.178 mmol, 85% yield) as a cream-coloured solid.

¹H NMR (δ, ppm, DMSO-d₆): 9.42 (d, 2H), 9, 9.38 (s, 1H), 9.06 (t, 1H), 8.77 (d, 1H), 8.66-8.57 (m, 1H), 8.44 (d, 2H), 8.33 (br. s, 2H), 8.17 (d, 2H), 3.70 (d, 2H), 2.29-2.14 (m, 4H), 1.99-1.77 (m, 2H). [ES MS] m/z: 378 (MH⁺).

### Example 3: N-((1-aminocyclobutyl)methyl)-4-(6-(5-fluoropyridin-3-yl)pyrazin-2-yl)benzamide, sulfuric acid salt

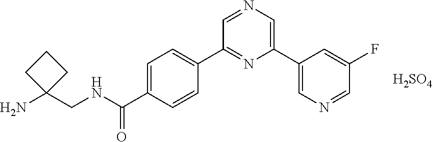

To a solution of tert-butyl (1-((4-(6-(5-fluoropyridin-3-yl)pyrazin-2-yl)benzamido)methyl)cyclobutyl)carbamate (100 mg, 0.209 mmol) in Dichloromethane (DCM) (0.5 mL) and Methanol (0.5 mL) at 40° C., sulfuric acid (0.1 mL, 1.876 mmol) was added dropwise, and the reaction was stirred at 40° C. The reaction was allowed to reach room temperature, tBuOMe was added and the solid was filtered and rinsed with tBuOMe (2×) to get after drying under a nitrogen stream an off-white solid. (125 mg, N74910-88-1).

¹H NMR (δ, ppm, DMSO-d₆): 9.44 (s, 1H), 9.43 (s, 1H), 9.39 (t, 1H), 8.92 (t, 1H), 8.79 (d, 1H), 8.66-8.58 (m, 1H), 8.49-8.45 (m, 2H), 8.13-8.08 (m, 2H), 8.06-7.94 (m, 3H), 3.67 (d, 2H), 2.27-2.11 (m, 4H), 1.97-1.77 (m, 2H). [ES MS] m/z: 378 (MH⁺).

### Example 4: N-((1-aminocyclobutyl)methyl)-4-(6-(5-fluoropyridin-3-yl)pyrazin-2-yl)benzamide

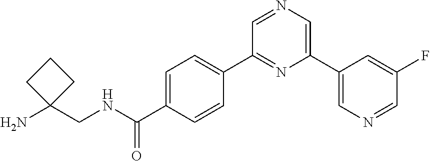 [SMILES: NC1(CNC(=O)c2ccc(-c3cncc(-c4cncc(F)c4)n3)cc2)CCC1]

To a suspension of tert-butyl (1-((4-(6-(5-fluoropyridin-3-yl)pyrazin-2-yl)benzamido)methyl)cyclobutyl)carbamate (74.27 g, 156 mmol) in dichloromethane (1 L) at room temperature, TFA (150 ml, 1947 mmol) was added dropwise over 30 min (it turned to a solution), the reaction was stirred at room temperature. After 16 h, water (1 L) was added and stirred for 15 min, the organic phase was separated, the aqueous phase was basified with saturated solution of Na₂CO₃, and a yellow solid appeared. The yellow solid was extracted with a mixture of DCM/MeOH 10% (3×700 ml). The organic phases were combined and washed with sat NaCl, and concentrated in vacuo to obtain a yellow solid, it was triturated with EtOH (50 ml) and filtered, rinsed with EtOH (25 ml) and dried to obtain titled compound (51 g, 135 mmol, 87%) as a crystalline yellowish solid.

¹H NMR (δ, ppm, DMSO-D6): 9.41 (d, 2H), 9.37 (t, 1H), 8.77 (d, 1H), 8.62-8.57 (m, 1H), 8.48-8.38 (m, 3H), 8.06 (d, 2H), 3.41 (d, 2H), 2.03 (m, 2H), 1.89 (s, 2H), 1.82-1.54 (m, 4H) [ES MS] m/z: 378 (MH⁺).

### Example 4a: Preparation of a Crystalline Form of N-((1-aminocyclobutyl)methyl)-4-(6-(5-fluoropyridin-3-yl)pyrazin-2-yl)benzamide

The X-ray powder diffraction (XRPD) pattern of the crystalline form provided in Example 4 is shown in FIG. 1, and a summary of the diffraction angles is given in Table 1 below. The XRPD analysis was conducted on a PANalytical Empyrean powder diffractometer, model 9430 060 03001 using a PIXcel3D detector. The acquisition conditions were: radiation: Cu Kα, generator tension: 45 kV, generator current: 40 mA, start angle: 2.0° 2θ, end angle: 40.0° 2θ, step size: 0.0263° 2θ, time per step: 46.665 seconds. The sample was prepared by mounting a few milligrams of sample on a silicon wafer (zero background plate), resulting in a thin layer of powder. The margin of error is approximately ±0.1° 2θ for each of the peak assignments. Peak intensities may vary from sample to sample due to preferred orientation.

TABLE 1

| Diffraction angle (°2θ) | d-spacings/Å |
|---|---|
| 6.7 | 13.3 |
| 11.2 | 7.9 |
| 12.7 | 7.0 |
| 13.4 | 6.6 |
| 16.2 | 5.5 |
| 16.6 | 5.3 |
| 17.9 | 4.9 |
| 20.9 | 4.3 |
| 26.7 | 3.3 |
| 28.2 | 3.2 |

### Intermediate 5: tert-butyl-(1-((4-(6-(5-fluoropyridin-3-yl)pyrazin-2-yl)benzamido)methyl)cyclopropyl)carbamate

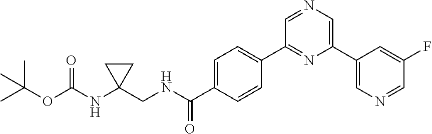 [SMILES: CC(C)(C)OC(=O)NC1(CNC(=O)c2ccc(-c3cncc(-c4cncc(F)c4)n3)cc2)CC1]

To a solution of 4-(6-(5-fluoropyridin-3-yl)pyrazin-2-yl)benzoic acid (300 mg, 1.016 mmol), tert-butyl (1-(aminomethyl)cyclopropyl)carbamate (Combi-Blocks, 246 mg, 1.321 mmol) and DIPEA (Sigma-Aldrich, 0.532 mL, 3.05 mmol) in N,N-Dimethylformamide (DMF) (10 mL) under N₂ atmosphere, HATU (Fluorochem, 541 mg, 1.422 mmol) was added and the resulting mixture was stirred at room temperature.

After 15.5 h, water was added until completed precipitation of a pale yellow solid (25 mL). The resulting solid was washed sequentially with 0.5M NaOH (25 mL) and water (2×25 mL) and then dried under air stream. This solid (450 mg) was purified by chromatographic column (solid charge in 3 g of SiO₂, 27 g SiO₂, Silicycle, Cy: EtOAc gradients from 80:20 to 0:100 in 30 min, Flow 30 mL/min) to give, after collecting and drying the appropriate fractions, the titled compound (358 mg, 0.772 mmol, 76% yield) as a white solid.

¹H NMR (δ, ppm, DMSO-d6): 9.41 (s, 1H), 9.40 (s, 1H), 9.37 (t, 1H), 8.77 (d, 1H), 8.63-8.56 (m, 1H), 8.52 (t, 1H), 8.45-8.39 (m, 2H), 8.04 (br. d, 2H), 7.26 (s, 1H), 3.43 (d, 2H), 0.83-0.76 (m, 2H), 0.67-0.61 (m, 2H). [ES MS] m/z: 464 (MH⁺).

### Example 5: N-((1-aminocyclopropyl)methyl)-4-(6-(5-fluoropyridin-3-yl)pyrazin-2-yl)benzamide, dihydrochloride

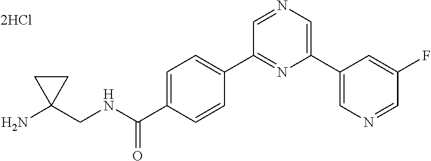 [SMILES: NC1(CNC(=O)c2ccc(-c3cncc(-c4cncc(F)c4)n3)cc2)CC1]

To a stirred solution of tert-butyl (1-((4-(6-(5-fluoropyridin-3-yl)pyrazin-2-yl)benzamido)methyl)cyclopropyl)carbamate (100 mg, 0.216 mmol) in dichloromethane (2 mL) HCl in 1,4 Dioxane (SYMAX LABORATORY, 4 N, 0.162 mL, 0.647 mmol) was added at 0° C. The reaction mixture was stirred at rt for 1 h.

The reaction mixture was diluted with DCM (5 mL) and excess solvent was concentrated under reduced pressure to get crude as brown solid. The crude was triturated with diethyl ether (3×10 mL) and the supernatant was decanted. Then, the compound was dried under reduced pressure to get titled compound (73 mg, 0.180 mmol, 77%) as a brown solid.

¹H NMR (δ, ppm, DMSO-d₆): 9.44 (s, 1H), 9.42 (s, 1H), 9.39 (m, 1H), 8.97 (t, 1H), 8.79 (d, 1H), 8.62 (m, 1H), 8.46 (d, 2H), 8.33 (br s, 3H), 8.13 (d, 2H), 3.58 (br d, 2H), 0.93 (s, 4H). [ES MS] m/z: 364 (MH⁺).

### Example 6: N-((1-aminocyclopropyl)methyl)-4-(6-(5-fluoropyridin-3-yl)pyrazin-2-yl)benzamide, ditrifluoroacetic acid salt

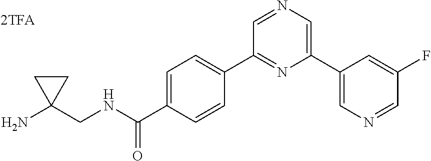 [SMILES: NC1(CNC(=O)c2ccc(-c3cncc(-c4cncc(F)c4)n3)cc2)CC1]

Tert-butyl (1-((4-(6-(5-fluoropyridin-3-yl)pyrazin-2-yl)benzamido)methyl)cyclopropyl) carbamate (358 mg, 0.772 mmol) was dissolved in trifluoroacetic acid (5 mL) at 0° C. and the reaction was allowed to reach room temperature. 14 h later, the reaction was concentrated under reduced pressure. The residue (500 mg) was dissolved in 3.6 mL of MeOH and purified by preparative HPLC (column: X-Bridge (50 mm×150 mm); Gradient: water (0.1% TFA): acetonitrile (0.1% TFA) 20% to 100% to 100% (8 min); 1 injection; Flow: 80 ml/min). The fractions containing product were collected and solvents evaporated in vacuo to yield 300 mg of fraction 1 and 155 mg of fraction 2 as colorless oils:
fraction 1 was suspended in ACN (10 mL) and water (10 mL) and lyophilized to give the titled compound (285 mg, 0.482 mmol, 62.4% yield) as a white solid.
fraction 2, was suspended in ACN (10 mL) and water (10 mL) and lyophilized to give titled compound (155 mg, 0.262 mmol, 33.9% yield) with lower purity (95% purity by HPLC) as a white solid.

¹H NMR (δ, ppm, DMSO-D6): 9.43 (s, 1H), 9.42 (s, 1H), 9.38 (t, 1H), 8.88 (t, 1H), 8.78 (d, 1H), 8.61 (m, 1H), 8.45 (m, 2H), 8.21 (br s, 3H), 8.08 (m, 2H), 3.58 (d, 2H), 0.91 (m, 4H). [ES MS] m/z: 364 (MH⁺).

### Intermediate 6: tert-butyl (1-(4-(6-(5-fluoropyridin-3-yl)pyrazin-2-yl)benzamido)-2-methylpropan-2-yl)carbamate

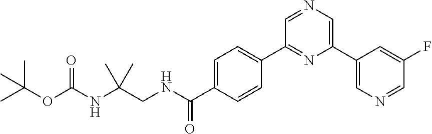 [SMILES: CC(C)(CNC(=O)c1ccc(-c2cncc(-c3cncc(F)c3)n2)cc1)NC(=O)OC(C)(C)C]

To a solution of 4-(6-(5-fluoropyridin-3-yl)pyrazin-2-yl)benzoic acid (187 mg, 0.633 mmol) and DIPEA (SIGMA-ALDRICH, 0.553 mL, 3.17 mmol) in N,N-Dimethylformamide (6.5 mL), HATU (FLUOROCHEM, 313 mg, 0.823 mmol) was added. After stirring 10 min, tert-butyl (1-amino-2-methylpropan-2-yl)carbamate (COMBI-BLOCKS, 155 mg, 0.823 mmol) was added and the resulting mixture was stirred at room temperature.

16.5 h later, the reaction was partitioned between 20 ml of 1N NH₄Cl and 50 mL EtOAc. The layers were separated and the organic was washed with 1N NH₄Cl (20 mL) and then with brine (20 mL). The resulting mixture was dried over anhydrous Na₂SO₄, filtered and concentrated under reduced pressure. The residue was purified by chromatographic column (loaded in 1.5 mL DCM, 12 g SiO₂, silicycle, Cy: EtOAc-EtOH 3/1 gradients form 100:0 to 85:15 to 50:50) to give, after collecting and concentrating the appropriate fractions, 250 mg of tert-butyl (1-(4-(6-(5-fluoropyridin-3-yl)pyrazin-2-yl)benzamido)-2-methylpropan-2-yl)carbamate (250 mg, 0.537 mmol, 85% yield) as a white solid.

¹H NMR (δ, ppm, DMSO-d6): 9.41 (d, 2H), 9.37 (t, 1H), 8.77 (d, 1H), 8.64 (t, 1H), 8.62-8.57 (m, 1H), 8.42 (d, 2H), 8.04 (d, 2H), 1H), 6.61 (br. s, 1H), 3.42 (d, 2H), 1.45-1.34 (m, 9H), 1.26 (s, 6H). [ES MS] m/z: 466 (MH⁺).

### Example 7: N-(2-amino-2-methylpropyl)-4-(6-(5-fluoropyridin-3-yl)pyrazin-2-yl)benzamide, ditrifluoroacetic acid salt

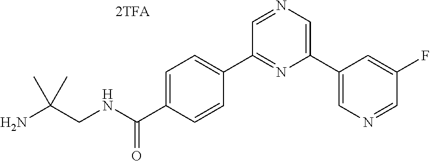 [SMILES: CC(C)(N)CNC(=O)c1ccc(-c2cncc(-c3cncc(F)c3)n2)cc1]

To a solution of tert-butyl (1-(4-(6-(5-fluoropyridin-3-yl)pyrazin-2-yl)benzamido)-2-methylpropan-2-yl)carbamate (295 mg, 0.634 mmol) in dry dichloromethane (2 mL) under N₂ atmosphere, trifluoroacetic acid (1.000 mL) was added and the resulting mixture was stirred overnight.

The reaction was diluted with MeOH and concentrated under reduced pressure. The residue was purified by preparative HPLC (sample loaded in 1.5 mL of MeOH, XBridge 30×100 mm, using as a mobile phase ACN:H2O (with 0.1% TFA), flow 35 mL/min collected and lyophilized to give 140 mg of N-(2-amino-2-methylpropyl)-4-(6-(5-fluoropyridin-3-yl)pyrazin-2-yl)benzamide, ditrifluoroacetic acid salt (140 mg, 0.292 mmol, 46.1% yield) as a white solid.

¹H NMR (δ, ppm, DMSO-D6): 9.42 (d, 2H), 9.38 (t, 1H), 8.88 (t, 1H), 8.77 (d, 1H), 8.63-8.56 (m, 1H), 8.48-8.41 (m, 2H), 8.14-8.06 (m, 2H), 7.89-7.73 (m, 1H), 7.88-7.73 (m, 1H), 3.53-3.40 (m, 2H), 1.38-1.20 (m, 6H) [ES MS] m/z: 366 (MH⁺).

### Intermediate 7: tert-butyl-((1R,2S)-2-(4-(6-(5-fluoropyridin-3-yl)pyrazin-2-yl)benzamido)cyclohexyl)carbamate

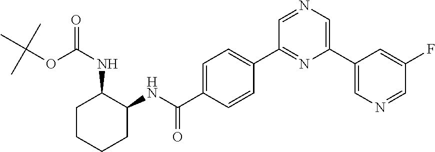 [SMILES: CC(C)(C)OC(=O)N[C@@H]1CCCC[C@@H]1NC(=O)c1ccc(-c2cncc(-c3cncc(F)c3)n2)cc1]

To a stirred solution of 4-(6-(5-fluoropyridin-3-yl)pyrazin-2-yl)benzoic acid (350 mg, 1.185 mmol) in N,N-Dimethylformamide (15 mL) was added DIPEA (Sonia, 0.621 mL, 3.56 mmol) and HATU (GLR, 676 mg, 1.778 mmol) at 0° C. The reaction mixture was stirred for 10 min at 0° C. Later tert-butyl ((1R,2S)-2-aminocyclohexyl)carbamate (TCI, 356 mg, 1.659 mmol) was added to the reaction.

The reaction mixture was stirred at room temperature for 16 h. Reaction mixture was quenched with cold water (50 mL) upon which a white solid crashed out. The obtained solid was filtered and washed with cold water (50 mL) and dried under vacuo. The crude was adsorbed into neutral alumina (1 g) and purified by isolera column chromatography (neutral alumina, 25 g snap) using 2% Methanol in dichloromethane as eluent. The product fractions were pooled and evaporated under vacuum to obtain the titled compound (430 mg, 0.865 mmol, 72.9% yield) as a brown solid.

¹H NMR (δ, ppm, DMSO-d₆): 9.42 (s, 1H), 9.41 (s, 1H), 9.37 (t, 1H), 8.77 (d, 1H), 8.63-8-55 (m, 1H), 8.41 (d, 2H), 8.00 (br. s, 3H), 6.69 (m, 1H), 4.08 (m, 1H), 3.80 (m, 1H), 1.78 (m, 2H), 1.56 (m, 4H), 1.32 (m, 11H). [ES MS] m/z: 392 [(MH⁺)−100].

### Example 8: N-((1S,2R)-2-aminocyclohexyl)-4-(6-(5-fluoropyridin-3-yl)pyrazin-2-yl)benzamide, dihydrochloride

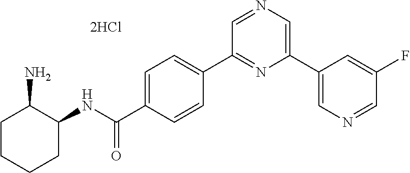 [SMILES: N[C@@H]1CCCC[C@@H]1NC(=O)c1ccc(-c2cncc(-c3cncc(F)c3)n2)cc1]

A solution of tert-butyl ((1R,2S)-2-(4-(6-(5-fluoropyridin-3-yl)pyrazin-2-yl)benzamido)cyclohexyl)carbamate (0.895 g, 1.821 mmol) in a mixture 1:9 of DCM: MeOH (10 mL) under N₂ atmosphere was heated at 40° C., and 37% hydrochloric acid (Sigma-Aldrich, 1 mL, 12.18 mmol) was added in one portion. The resulting mixture (solution) was stirred at 40° C. overnight.

After 20 h, the reaction was concentrated under reduced pressure. The resulting solid was triturated with TBME but the solid formed was not filterable. After concentration, the mixture was triturated with iPrOH but again the solid formed was not filterable.

After concentrating again the mixture was dissolved in MeOH/DCM mixtures and DCM was evaporated slowly in the rotavap. The resulting suspension was stored in the fridge for 2 days. The suspension was filtered and washed with cool MeOH. The resulting solid was dried under air steam to give 800 mg of N-((1S,2R)-2-aminocyclohexyl)-4-(6-(5-fluoropyridin-3-yl)pyrazin-2-yl)benzamide, dihydrochloride (800 mg, 1.723 mmol, 95% yield) as a white solid.

¹H NMR (δ, ppm, DMSO-d6): 9.43 (s, 1H), 9.41 (s, 1H), 9.38 (t, 1H), 8.78 (d, 1H), 8.61 (m, 1H), 8.42 (d, 2H), 8.39 (d, 1H), 8.17 (d, 2H), 8.12 (br d, 3H), 4.33 (td, 1H), 3.48 (m, 1H), 1.90 (m, 2H), 1.67 (m, 4H), 1.42 (m, 2H).). [ES MS] m/z: 392 [(MH⁺)].

### Example 9: N-((1S,2R)-2-aminocyclohexyl)-4-(6-(5-fluoropyridin-3-yl)pyrazin-2-yl)benzamide, ditrifluoroacetic acid salt

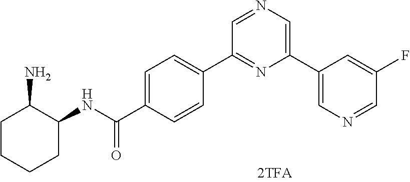 [SMILES: N[C@@H]1CCCC[C@@H]1NC(=O)c1ccc(-c2cncc(-c3cncc(F)c3)n2)cc1]

To a suspension of N-((1S,2R)-2-aminocyclohexyl)-4-(6-(5-fluoropyridin-3-yl)pyrazin-2-yl)benzamide (850 mg, 2.171 mmol) in dichloromethane (DCM) (40 mL), trifluoroacetic acid (2 mL, 26.0 mmol) was added (it turned a solution) and stirred at rt for 1 h.

The crude was concentrated in vacuo and the resultant solid was dry in a vacuum oven at 40° C. for 18 h to obtain a yellowish solid N-((1S,2R)-2-aminocyclohexyl)-4-(6-(5-fluoropyridin-3-yl)pyrazin-2-yl)benzamide, ditrifluoroacetic acid salt (1.36 g, 2.195 mmol, approximately 99% yield).

¹H NMR (δ, ppm, DMSO-d₆): 9.44 (s, 1H), 9.42 (s, 1H), 9.38 (m, 1H), 8.78 (d, 1H), 8.60 (m, 1H), 8.43 (d, 2H), 8.23 (d, 1H), 8.12 (d, 2H), 7.82 (br d, 3H), 4.37 (m, 1H), 3.45 (m, 1H), 1.75 (m, 6H), 1.42 (m, 2H). [ES MS] m/z: 392 (MH⁺).

### Example 10: N-((1S,2R)-2-aminocyclohexyl)-4-(6-(5-fluoropyridin-3-yl)pyrazin-2-yl)benzamide

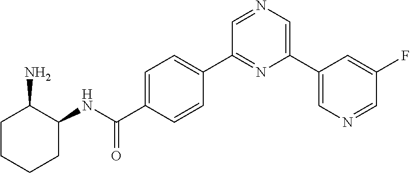 [SMILES: N[C@@H]1CCCC[C@@H]1NC(=O)c1ccc(-c2cncc(-c3cncc(F)c3)n2)cc1]

To a solution of tert-butyl ((1R,2S)-2-(4-(6-(5-fluoropyridin-3-yl)pyrazin-2-yl)benzamido)cyclohexyl)carbamate (1.7 g, 3.46 mmol) in a mixture of Dichloromethane (DCM) (5 mL) and Methanol (5.00 mL), 6N hydrochloric acid in iPrOH (FLUOROCHEM, 1.32 mL, 7.92 mmol) was added and stirred at rt. After 16 h, the solid was filtered, the solid was suspended in water and Na₂CO₃ sat solution was added until a basic pH was reached, the solid was extracted with DCM/MeOH 10% (3×10 ml), the organic phases were put together, washed with NaCl sat, dried over Na₂SO₄, filtered and concentrated in vacuo to provide a yellow crystalline solid N-((1S,2R)-2-aminocyclohexyl)-4-(6-(5-fluoropyridin-3-yl)pyrazin-2-yl)benzamide (1.02 g, 2.61 mmol, 75% yield).

¹H NMR (δ, ppm, DMSO-d₆): 9.41 (s, 2H), 9.37 (m, 1H), 8.77 (d, 1H), 8.60 (m, 1H), 8.40 (d, 2H), 8.03 (d, 2H), 8.01 (d, 1H), 3.95 (m, 1H), 3.06 (m, 1H), 1.84-1.46 (m, 8H), 1.32 (m, 2H). [ES MS] m/z: 392 (MH⁺).

### Example 10a: Crystalline Form of N-((1S,2R)-2-aminocyclohexyl)-4-(6-(5-fluoropyridin-3-yl)pyrazin-2-yl)benzamide

The X-ray powder diffraction (XRPD) pattern of the crystalline form provided in Example 11 is shown in FIG. 3 and a summary of the diffraction angles is given in Table 2 below. The XRPD analysis was conducted on a PANalytical Empyrean powder diffractometer, model 9430 060 03001 using a PIXcel3D detector. The acquisition conditions were: radiation: Cu Kα, generator tension: 45 kV, generator current: 40 mA, start angle: 2.0° 2θ, end angle: 40.0° 2θ, step size: 0.0263° 2θ, time per step: 46.665 seconds. The sample was prepared by mounting a few milligrams of sample on a silicon wafer (zero background plate), resulting in a thin layer of powder. The margin of error is approximately ±0.1° 2θ for each of the peak assignments. Peak intensities may vary from sample to sample due to preferred orientation.

TABLE 2

| Diffraction angle (°2θ) | d-spacings/Å |
|---|---|
| 5.4 | 16.3 |
| 10.8 | 8.2 |
| 15.3 | 5.8 |
| 16.6 | 5.4 |
| 18.2 | 4.9 |
| 20.1 | 4.4 |
| 21.8 | 4.1 |
| 22.4 | 4.0 |
| 28.1 | 3.2 |
| 31.7 | 2.8 |

### Biological and Physicochemical Data

The compounds of this invention may be tested in one of several biological and physicochemical assays to determine the concentration of the compound which is required to provide a given pharmacological effect. The assays are described below.

### In Vitro Potency

P. falciparum Growth Inhibition Assay Sybr Green

The parasite growth is determined measuring the staining of the DNA's minor groove with SYBR Green I, which labels the remaining parasites. The parasite growth is determined measuring the staining of the DNA's minor groove with SYBR Green I, which labels the remaining parasites. The DNA-SYBR complex absorbs light reaching the peak at λ=498 nm and emits at a peak of λ=522 nm.

### Methodology

Briefly, a culture of 3D7A strain parasitized red blood cells (RBC) (0.5% parasitemia and 2% haematocrit in RPMI-1640 supplemented with 25 ml albumax and 150 mM hypoxanthine for 500 ml) is exposed to 3-fold serial dilutions of the compound. Plates are incubated 48 h at 37° C., 5% CO2, 5% O2, 90% N2. At 48 h of incubation, SyberGreen I solution (2×) is added and plates are incubated for 30 minutes at 37° C., 5% CO2, 5% O2, 90% N2 and then plates are frozen at −80° C. Then plates are read in the EnVision (EnVision Multilabel Plate Reader, Perkin Elmer). Data (pIC50) are generated by ActivityBase (IDBS) and graphs are checked by Spotfire.

### Results

The results are shown in Table 3 below. Inhibition of parasite growth in whole cell assay (infected human eritrocites) in vitro is directly linked to reduction of parasitemia in vivo.

TABLE 3

| Example | Pf3D7 pIC₅₀ | Average value pIC₅₀ |
|---|---|---|
| 1 | 8.48-8.60 | 8.50 |
| 2 | 8.47-8.63 | 8.50 |
| 3 | 8.30-8.60 | 8.50 |
| 4 | 8.54-8.76 | 8.60 |
| 5 | 8.34-8.60 | 8.50 |
| 7 | 8.05-8.15 | 8.10 |
| 8 | 8.33-8.6 | 8.40 |
| 9 | 8.53-8.60 | 8.60 |
| 10 | 8.42-8.51 | 8.50 |

### In Vivo Efficacy

P. falciparum In Vivo Efficacy Assay.

Antimalarial in vivo efficacy was determined using the P. falciparum mouse model following the procedure described in: Jimenez-Díaz, M. B., Mulet, T., Viera, S., Gómez, V., Garuti, H., Ibañez, J., Alvarez-Doval, A., Shlutz, D. L., Martinez, A., Improved Murine Model Of Malaria Using Plasmodium falciparum (Competent Strains and Non-Myelodepleted NOD-scid IL2R_null Mice Engrafted with Human Erythrocytes) Antimicrob. Agents Chemother 2009, 53 (10), 4533-4536.

### Results

The goal of the study was to assess the therapeutic efficacy of the examples against Plasmodium falciparum Pf3D7^{0087/N9} growing in peripheral blood of NODscidIL2Ry^{0087/N9} mice engrafted with human erythrocytes. The levels of the examples were measured in serial peripheral blood samples obtained from each mouse of the efficacy experiment during the first 23 hrs after the first dose. The area under the curve of levels of compound is used to estimate the exposure in blood during the first 23 hours after the first administration (AUC₀₋₂₃ₕ).

The effect of Examples 1, 2, 5, 7, 8 and 10 on P. Falciparum Pf3D7^{0087/N9} was assessed by microscopy and flow cytometry.

The results are shown in Table 4 below.

TABLE 4

| | ED90 mean | AUC_{ED90} mean |
|---|---|---|
| Compound | (mg · Kg⁻¹) | (mg · h · mL⁻¹ · day⁻¹)* |
| Example 1 | 1.28 | 0.067 |
| Example 2 | 1.14 | 0.121 |
| Example 5 | 1.04 | 0.13 |
| Example 7 | 1.74 | 0.05 |
| Example 8 | 1.59 | 0.26 |
| Example 10 | 0.95 | 0.20 |

*Interval of confidence 95%

### Solubility Assessment in Biorelevant Media

Compounds' equilibrium solubility were measured at room temperature after 4 hour equilibration in Fasted State Simulated Intestinal Fluid (FaSSIF) at pH 6.5.

### Solvents and Buffers

Organic solvents of HPLC grade were used. Ultra pure water (Milli-Q grade) was used. Buffers were prepared with ultra pure water.

### Procedure

a) 1 mg of solid compound was weighed in a 4 mL glass vial and 1 mL of the FaSSIF was added.
b) 1 mg of the same solid was weighed in a 4 mL glass vial and 1 ml of dimethyl sulfoxide (DMSO) was added to give a standard solution of known concentration.
c) The FaSSIF sample was left on a shaker (900 rpm) for 4 hours at room temperature.
d) After 4 hours the resulting suspension was transferred to a Multiscreen HTS-PCF 96-well solubility filter plate and vacuum filtration was used to separate the residual solid and the filtrate.
e) The filtrate was analysed by LC-UV.
f) A set of 3 internal standards of known solubility (Atovaquone, Nimesulide and Warfarin of 2, 20 and 140 mg/mL respectively) were tested using the same procedure alongside the compounds to assess the suitability of the process

### LC-UV Assay for Analytical Quantification

Quantification of the compound concentration in the filtrate was performed by HPLC-UV using single point calibration of a known concentration of the compound in DMSO. The dynamic range of the assay was 1-1000 μg/ml.

### Analysis of Data

The analysis of the LC-UV data was performed using an in house Excel macro. The solubility (mg/mL) for each compound was calculated from the peak areas of the sample and standard.

### Results

Results are shown in Table 5 below. The aqueous solubility of a compound is an essential drug characteristic influencing oral bioavailability. FaSSIF media simulate the fluids in the intestinal region from which most drugs are absorbed. Values with limited drug solubility in the intestine may lead to incomplete and variable drug absorption. It is, therefore, of interest to estimate the amount of dissolved drug in the gastrointestinal tract.

TABLE 5

| Compound | FaSSIF solubility (μg/mL) |
|---|---|
| EXAMPLE 1 | 429 |
| EXAMPLE 2 | 398 |
| EXAMPLE 4 | >1000 |
| EXAMPLE 5 | 380 |
| EXAMPLE 6 | 339 |
| EXAMPLE 7 | 742 |
| EXAMPLE 8 | >1000 |
| EXAMPLE 9 | 789 |
| EXAMPLE 10 | 941 |

### Solubility Assessment in Aqueous Buffer

The kinetic aqueous solubility was measured at room temperature after 1 hour equilibration in aqueous phosphate buffered saline (PBS) at pH7.4, and is determined by measuring the concentration of solute in solution after precipitation from DMSO stock solution.

### Solvents and Buffers

Organic solvents of HPLC grade were used. Ultra pure water (Milli-Q grade) was used. Buffers were prepared with ultra pure water.

### Procedure

5 ml of 10 mM DMSO stock solution was dispensed in the well of a Multiscreen HTS-PCF 96-well solubility filter plate and 95 ml of PBS was added.

The sample plate was left to equilibrate for 1 hour at room temperature.

Vacuum filtration was applied to filter the solution into a 96-well collection plate.

The filtrate was analysed by HPLC-CAD (Charged Aerosol Detector).

Calibration standards of Ketoconazole and Primidone were prepared by serial dilutions in DMSO at concentrations ranging from 0.016 to 4.5 mg/ml to produce the calibration curve.

### LC-UV Assay for Analytical Quantification

Quantification of the compound concentration in the filtrate was performed by HPLC-CAD using sample peaks areas from the CAD and the calibration coefficients.

### Analysis of Data

The analysis of the HPLC-CAD data was performed using an in house Excel macro. The concentration (mM) and solubility (μg/ml) for each compound was calculated from the peak areas of the sample and calibration coefficients.

### Results

The results are shown in Table 6 below.

TABLE 6

| Compound | Concentration (mM) | Solubility (μg/mL) |
|---|---|---|
| EXAMPLE 1 | ≥329 | ≥124.2 |
| EXAMPLE 2 | ≥335 | ≥126.4 |
| EXAMPLE 3 | ≥251 | ≥94.7 |
| EXAMPLE 4 | ≥344 | ≥129.8 |
| EXAMPLE 5 | ≥313 | ≥113.7 |
| EXAMPLE 6 | ≥549 | ≥199.5 |
| EXAMPLE 7 | ≥231 | ≥84.4 |
| EXAMPLE 8 | ≥271 | ≥106.1 |
| EXAMPLE 9 | 293 | 114.7 |
| EXAMPLE 10 | ≥348 | ≥136.2 |

1. A compound of Formula (I): 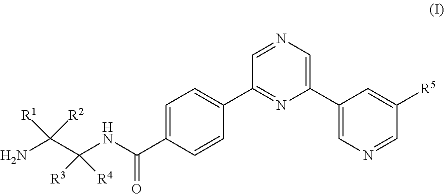
or a pharmaceutically acceptable salt or stereoisomer thereof, wherein:
R¹, R², R³, and R⁴ are each independently selected from the group consisting of H, halo, C₁-C₆ alkyl, and 3- to 7-membered cycloalkyl ring optionally containing a heteroatom selected from the group consisting of O, S, SO, SO₂, NH, and N—C₁-C₆ alkyl; or
R¹ and R² are together with the atom to which they are simultaneously attached form a 3-, 4-, 5-, 6-, or 7-membered cycloalkyl ring optionally containing a heteroatom selected from the group consisting of O, S, SO, SO₂, NH, and N—C₁-C₆ alkyl, wherein R³ and R⁴ are each independently selected from the group consisting of H, C₁-C₆ alkyl, and 3- to 7-membered cycloalkyl ring optionally containing a heteroatom selected from the group consisting of O, S, SO, SO₂, NH, and N—C₁-C₆ alkyl; or
R¹ and R³ are together with the atoms to which they are simultaneously attached form a 3-, 4-, 5-, 6-, or 7-membered cycloalkyl ring optionally containing a heteroatom selected from the group consisting of O, S, SO, SO₂, NH, and N—C₁-C₆ alkyl, wherein R² and R⁴ are each independently selected from the group consisting of H, C₁-C₆ alkyl, and 3- to 7-membered cycloalkyl ring optionally containing a heteroatom selected from the group consisting of O, S, SO, SO₂, NH, and N—C₁-C₆ alkyl; and
R⁵ is selected from the group consisting of halo and C₁-C₆ alkyl.

2. The compound or pharmaceutically acceptable salt or stereoisomer thereof according to claim 1, wherein R⁵ is halo.

3. The compound or pharmaceutically acceptable salt or stereoisomer thereof according to claim 1, wherein R⁵ is F.

4. The compound or pharmaceutically acceptable salt or stereoisomer thereof according to claim 1, wherein R³ and R⁴ are each H.

5. The compound or pharmaceutically acceptable salt or stereoisomer thereof according to claim 1, wherein R¹ and R² are together with the atom to which they are simultaneously attached form a 3-, 4-, 5-, 6-, or 7-membered cycloalkyl ring.

6. The compound or pharmaceutically acceptable salt or stereoisomer thereof according to claim 1, wherein the compound is

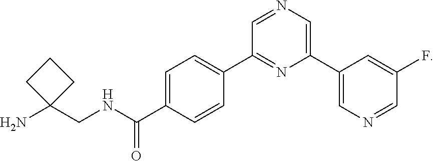 [SMILES: NC1(CNC(=O)c2ccc(-c3cncc(-c4cncc(F)c4)n3)cc2)CCC1]

7. The compound according to claim 6, wherein the compound is in the form of a free base.

8. The compound according to of claim 7, wherein the compound has
i) an X-ray powder diffraction pattern (XRPD) substantially as shown in FIG. 1; and/or
ii) an X-ray powder diffraction pattern (XRPD) with specific peaks at 2θ values, ±0.1° 2θ experimental error, of 6.7, 11.2, 12.7, 13.4, 16.2, 16.6, 17.9, 20.9, 26.7, and 28.2 degrees.

9. The compound according to claim 6, wherein the compound is in the form of a pharmaceutically acceptable sulfuric acid.

10. The compound according to claim 6, wherein the compound is in the form of a pharmaceutically acceptable dihydrochloride.

11. The compound according to claim 6, wherein the compound is in the form of a pharmaceutically acceptable ditrifluoroacetic acid salt.

12. The compound or pharmaceutically acceptable salt or stereoisomer thereof according to claim 1, wherein the compound is

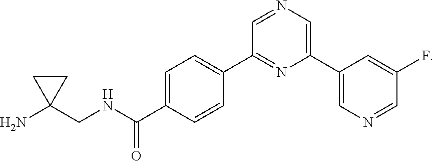 [SMILES: NC1(CNC(=O)c2ccc(-c3cncc(-c4cncc(F)c4)n3)cc2)CC1]

13. The compound or pharmaceutically acceptable salt or stereoisomer thereof according to claim 1, wherein R¹ and R² are each C₁-C₆ alkyl.

14. The compound or pharmaceutically acceptable salt or stereoisomer thereof according to claim 1, wherein the compound is

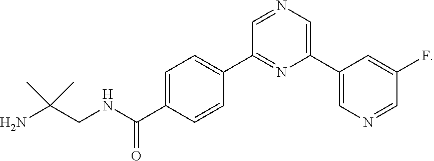 [SMILES: CC(C)(N)CNC(=O)c1ccc(-c2cncc(-c3cncc(F)c3)n2)cc1]

15. The compound or pharmaceutically acceptable salt or stereoisomer thereof according to claim 1, wherein R¹ and R³ are together with the atoms to which they are simultaneously attached form a 3-, 4-, 5-, 6-, or 7-membered cycloalkyl ring.

16. The compound or pharmaceutically acceptable salt or stereoisomer thereof according to claim 1, wherein the compound is

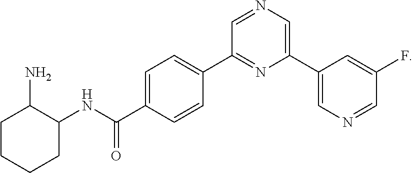 [SMILES: NC1CCCCC1NC(=O)c1ccc(-c2cncc(-c3cncc(F)c3)n2)cc1]

17. The compound according to claim 16, wherein the compound is in the form of a free base.

18. The compound according to claim 17, wherein the compound has
i) an X-ray powder diffraction pattern (XRPD) substantially as shown in FIG. 2; and/or
ii) an X-ray powder diffraction pattern (XRPD) with specific peaks at 2θ values, ±0.1° 2θ experimental error, of 5.4, 10.8, 15.3, 16.6, 18.2, 20.1, 21.8, 22.4, 28.1, and 31.7 degrees.

19. A compound or pharmaceutically acceptable salt or stereoisomer thereof selected from the group consisting of N-((1-aminocyclobutyl)methyl)-4-(6-(5-fluoropyridin-3-yl)pyrazin-2-yl)benzamide, N-(2-amino-2-methylpropyl)-4-(6-(5-fluoropyridin-3-yl)pyrazin-2-yl)benzamide, N-(2-aminocyclohexyl)-4-(6-(5-fluoropyridin-3-yl)pyrazin-2-yl)benzamide, and N-((1-aminocyclopropyl)methyl)-4-(6-(5-fluoropyridin-3-yl)pyrazin-2-yl)benzamide.

20. A pharmaceutical composition comprising (a) the compound or pharmaceutically acceptable salt or stereoisomer thereof according to claim 1, and (b) a pharmaceutically acceptable excipient.

21. (canceled)

22. (canceled)

23. (canceled)

24. (canceled)

25. A method of treating a parasitic protozoal infection in a human comprising administering to the human a therapeutically effective amount of the compound or pharmaceutically acceptable salt or stereoisomer thereof according to claim 1.

26. A combination comprising (a) the compound or pharmaceutically acceptable salt or stereoisomer thereof according to claim 1, and (b) at least one other anti-malarial agent.

27. A method of treating a parasitic protozoal infection in a human comprising administering to the human a therapeutically effective amount of the combination according to claim 25.

28. The method according to claim 25, wherein the parasitic protozoal infection is malaria.

29. The method according to claim 25, wherein the parasitic protozoal infection is Plasmodium falciparum.

30. A method of treating a parasitic protozoal infection in a human comprising administering to the human a therapeutically effective amount of the pharmaceutical composition according to claim 20.

31. The method according to claim 30, wherein the parasitic protozoal infection is malaria.

32. The method according to claim 30, wherein the parasitic protozoal infection is Plasmodium falciparum.
